(19) United States
(12) Patent Application Publication
(10) Pub. No.: US 2006/0058324 A1
(43) Pub. Date: Mar. 16, 2006

(54) 4-Amino-5-phenyl-7-cyclohexyl-pyrrolo 2,3-d pyrimidine derivatives

(76) Inventors: Hans-Georg Capraro, Rheinfelden (CH); Pascal Furet, Thann (FR); Carlos Garcia-Echeverria, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/534,427

(22) PCT Filed: Nov. 11, 2003
(86) PCT No.: PCT/EP03/12594

§ 371 Date: May 10, 2005

(30) Foreign Application Priority Data
Nov. 12, 2002 (GB) 0226370.5

(51) Int. Cl. A61K 31/519; C07D 487/02

(52) U.S. Cl. 514/265.1; 544/280

(57) ABSTRACT

The invention relates to new 4-amino-5-phenyl-7-cyclohexyl-pyrrolo[2,3-d]pyrimidine derivatives, processes for the preparation thereof, the application thereof in a process for the treatment of the human or animal body, the use thereof, alone or in combination with one or more other pharmaceutically active compounds, for the treatment of a disease, especially a proliferative disease, such as a tumour disease, a method for the treatment of such diseases in mammals, especially in humans, and the use of such a compound, alone or in combination with one or more other pharmaceutically active compounds, for the preparation of a pharmaceutical composition (medicament) for the treatment especially of a proliferative disease, such as a tumour.

The invention relates to new 4-amino-5-phenyl-7-cyclohexyl-pyrrolo[2,3-d]pyrimidine derivatives, processes for the preparation thereof, the application thereof in a process for the treatment of the human or animal body, the use thereof—alone or in combination with one or more other pharmaceutically active compounds—for the treatment of a disease, especially a proliferative disease, such as a tumour disease, a method for the treatment of such diseases in mammals, especially in humans, and the use of such a compound—alone or in combination with one or more other pharmaceutically active compounds—for the preparation of a pharmaceutical composition (medicament) for the treatment especially of a proliferative disease, such as a tumour.

Surprisingly, it has now been found that the compounds of formula I, described below, are potent inhibitors of the tyrosine kinase activity of the Insulin-like growth factor I receptor (IGF-IR) and inhibit IGF-IR-dependent cell proliferation. The presence of the substituents, preferably benzyloxy substituents, at position 3 of the phenyl group of the 4-amino-5-phenyl-7-cyclohexyl-pyrrolo[2,3-d]pyrimidine scaffold together with the presence of the substitutent R₂ as defined herein below is crucial for the efficacy and/or the specificity of the compounds of the present invention as IGF-IR tyrosine kinase inhibitors and their potential and/or selectivity to inhibit IGF-IR-dependent cell proliferation.

The compounds of formula I permit, for example, an unexpected new therapeutic approach, especially for diseases in the treatment of which, and also for the prevention of which, an inhibition of the IGF-IR tyrosine kinase and/or of the IGF-IR-dependent cell proliferation shows beneficial effects. Such diseases include proliferative diseases, such as tumours, like for example breast, renal, prostate, colorectal, thyroid, ovarian, pancreas, neuronal, lung (small cell lung cancer or non-small cell lung cancer), uterine and gastrointestinal tumours as well as retinoblastomas, osteosarcomas, melanomas and hematologic malignancies such as B- and T-cell acute lymphoblastic leukemia, acute and chronic myeloid leukemia and multiple myeloma.

The Invention relates to compounds of formula I 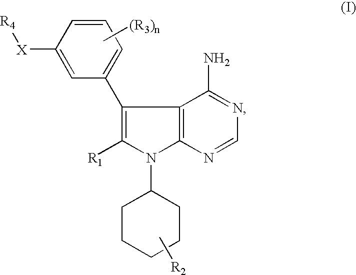
wherein
n is from 0 to 4,
R₁ is hydrogen, unsubstituted or substituted lower alkyl or halogen,
R₂ is hydroxy; unsubstituted, mono- or disubstituted amino; a heterocyclic radical containing at least one nitrogen ring atom and being attached to the cyclohexane ring of the molecule of formula I via a nitrogen ring atom; a radical R₅—(C═Y)—NH—, wherein R₅ is unsubstituted or substituted lower alkyl, unsubstituted, mono- or disubstituted amino, a heterocyclic radical, or etherified hydroxy, and Y is oxygen, sulfur or imino; or a radical R₆-sulfonylamino, wherein R₆ is unsubstituted or substituted lower alkyl, unsubstituted, mono- or disubstituted amino or phenyl optionally substituted by lower alkyl, lower alkoxy or nitro,
R₃ is lower alkyl, hydroxy-, amino- or halogen-substituted lower alkyl, hydroxy, cyano, lower alkoxy, lower alkanoyl, lower alkanoyloxy, amino, mono- or di-lower alkylamino, lower alkanoylamino, carboxy, lower alkoxycarbonyl or halogen, wherein the R₃ substituents can be selected independently of one another if n>1,
R₄ is a radical R₇—CR₈(R₉)—, wherein R₇ is cyclobutyl, cyclopentyl, cyclohexyl, phenyl, furyl, pyrrolyl, thienyl or pyridyl, said R₇ substitutents being optionally substituted by one or more radicals selected from lower alkyl and halogen, and R₈ and R₉ are independently of each other hydrogen, lower alkyl or halogen, and
X is selected from —O—, —NH— and —S—,
or a salt thereof.

The general terms used hereinbefore and hereinafter preferably have within the context of this disclosure the following meanings, unless otherwise indicated:

Where compounds of formula I are mentioned, this is meant to include also the tautomers and N-oxides of the compounds of formula I.

Where the plural form is used for compounds, salts, and the like, this is taken to mean also a single compound, salt, or the like.

Asymmetric carbon atoms of a compound of formula I that are optionally present may exist in the (R), (S) or (R,S) configuration, preferably in the (R) or (S) configuration. Substituents at a double bond or a ring may be present in cis- (=Z-) or trans (=E-) form. The compounds may thus be present as mixtures of isomers or as pure isomers, preferably as enantiomer-pure diastereomers.

The prefix “lower” denotes a radical having up to and including a maximum of 7, especially up to and including a maximum of 4 carbon atoms, the radicals in question being either un-branched or branched with single or multiple branching.

Lower alkyl is, for example, methyl, ethyl, n-propyl, isopropyl, n-butyl, isobutyl, sec-butyl, tert-butyl, n-pentyl, isopentyl, neopentyl, n-hexyl or n-heptyl.

Lower alkyl R₅ is preferably methyl.

Lower alkyl R₆ is preferably methyl.

Substituted lower alkyl is lower alkyl as defined above where one or more, preferably one, substituents may be present, such as amino, N-lower alkylamino, N,N-di-lower alkylamino, N-lower alkanoylamino, N,N-di-lower alkanoylamino, hydroxy, lower alkoxy, lower alkoxy-lower alkoxy, lower alkanoyl, lower alkanoyloxy, cyano, nitro, carboxy, lower alkoxycarbonyl, carbamoyl, amidino, guanidino, ureido, mercapto, lower alkylthio, halogen or a heterocyclic radical.

Substituted lower alkyl R₅ is preferably lower alkyl, especially methyl, substituted by a heterocyclic radical.

Halogen is primarily fluorine, chlorine, bromine, or iodine, especially fluorine, chlorine, or bromine.

Mono- or disubstituted amino is amino substituted by one or two radicals selected independently of one another from e.g.: unsubstituted or substituted lower alkyl; phenyl or phenyl-lower alkyl wherein the phenyl radical is optionally substituted by e.g. unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkoxy-lower alkoxy, lower alkanoyl, lower alkanoyloxy, cyano, nitro, carboxy, lower alkoxycarbonyl, carbamoyl, amidino, guanidino, ureido, mercapto, lower alkylthio or halogen; adamantanyl; and a heterocyclic radical.

Monosubstituted amino R₂ preferably represents pyrimidinyl-amino, 1,4,5,6-tetrahydro-pyrimidinyl-amino or 4,5-dihydro-1H-imidazolyl-amino.

Disubstituted amino R₂ preferably represents N,N-di-lower alkylamino.

Monosubstituted amino R₅ is preferably N-lower alkylamino, wherein the lower alkyl moiety is optionally substituted by phenyl, lower alkyl-phenyl, lower alkoxy-phenyl, morpholinyl or N,N-di-lower alkylamino.

Disubstituted amino R₆ is preferably N,N-di-lower alkylamino.

A heterocyclic radical contains especially up to 20 carbon atoms and is preferably a saturated or unsaturated monocyclic radical having from 4 or 8 ring members and from 1 to 3 heteroatoms which are preferably selected from nitrogen, oxygen and sulfur, or a bi- or tricyclic radical wherein, for example, one or two benzene radicals are annellated (fused) to the mentioned monocyclic radical. Preferred above all, the heterocyclic radical contains at least one nitrogen ring atom whereby the binding of the heterocyclic radical to the radical of the molecule of formula I occurs via a nitrogen ring atom. The heterocyclic radical is optionally substituted by one or more, preferably by one or two, radicals such as e.g. unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkoxy-lower alkoxy, lower alkanoyl, lower alkanoyloxy, cyano, nitro, carboxy, lower alkoxycarbonyl, carbamoyl, amidino, guanidino, ureido, mercapto, lower alkylthio, halogen, phenyl or pyridyl. Most preferably a heterocyclic radical is azetidinyl, pyrrolidinyl, piperidyl, azepanyl, piperazinyl, tetrahydropyranyl, morpholinyl or thiomorpholinyl, wherein said radicals are optionally substituted by one or more, preferably one or two, radicals selected independently of one another from the group consisting of lower alkyl, hydroxy-lower alkyl, free or etherified hydroxy, lower alkoxycarbonyl, carbamoyl, phenyl and pyridyl and the binding of the heterocyclic radical to the radical of the molecule of formula I occurs via a nitrogen ring atom.

A heterocyclic radical R₂ is as defined above for a heterocyclic radical with the proviso that it contains at least one nitrogen ring atom whereby the binding of the heterocyclic radical R₂ to the cyclohexane ring of the molecule of formula I occurs via a nitrogen ring atom.

A heterocyclic radical R₂ containing at least one nitrogen ring atom and being attached to the cyclohexane ring of the molecule of formula I via a nitrogen ring atom preferably represents azetidinyl, pyrrolidinyl, piperidyl, lower alkyl-piperazinyl, morpholinyl or thiomorpholinyl.

In R₅ being lower alkyl substituted by a heterocyclic radical, the heterocyclic radical preferably represents piperidyl, lower alkyl-piperazinyl or morpholinyl.

Etherified hydroxy is, for example, alkoxy, especially lower alkoxy. The lower alkyl moiety of lower alkoxy is optionally substituted by one or more, preferably one, radicals such as e.g. amino, N-lower alkylamino, N,N-di-lower alkylamino, N-lower alkanoylamino, N,N-di-lower alkanoylamino, hydroxy, lower alkoxy, lower alkoxy-lower alkoxy, lower alkanoyl, lower alkanoyloxy, cyano, nitro, carboxy, lower alkoxycarbonyl, carbamoyl, amidino, guanidino, ureido, mercapto, lower alkylthio, halogen or a heterocyclic radical.

Etherified hydroxy R₅ is preferably lower alkoxy wherein the lower alkyl moiety is optionally substituted by lower alkoxy.

n is preferably 0.

R₁ preferably represents hydrogen, lower alkyl or halogen (especially bromine), most preferably lower alkyl, especially methyl.

R₄ is preferably benzyl.

X is preferably —O—.

Y is preferably oxygen.

Salts are especially the pharmaceutically acceptable salts of compounds of formula I (or an N-oxide thereof).

Such salts are formed, for example, as acid addition salts, preferably with organic or inorganic acids, from compounds of formula I (or an N-oxide thereof) with a basic nitrogen atom, especially the pharmaceutically acceptable salts.

In the presence of negatively charged radicals, such as carboxy or sulfo, salts may also be formed with bases, e.g. metal or ammonium salts, such as alkali metal or alkaline earth metal salts, or ammonium salts with ammonia or suitable organic amines, such as tertiary monoamines.

In the presence of a basic group and an acid group in the same molecule, a compound of formula I (or an N-oxide thereof) may also form internal salts.

For isolation or purification purposes it is also possible to use pharmaceutically unacceptable salts, for example picrates or perchlorates. Only the pharmaceutically acceptable salts or free compounds (if the occasion arises, in the form of pharmaceutical compositions) attain therapeutic use, and these are therefore preferred.

In view of the close relationship between the novel compounds in free form and in the form of their salts, including those salts that can be used as intermediates, for example in the purification or identification of the novel compounds, hereinbefore and hereinafter any reference to the free compounds is to be understood as referring also to the corresponding salts, as appropriate and expedient.

The compounds of formula I have valuable pharmacological properties, as described herein-before and hereinafter.

The efficacy of the compounds of the invention as inhibitors of IGF-IR tyrosine kinase activity can be demonstrated using a cellular “Capture ELISA”. In this assay the activity of the compounds of the invention against Insulin-like growth factor I (IGF-I) induced autophosphorylation of the IGF-IR is determined. The assay is conducted as follows:

For the assay NIH-3T3 mouse fibroblasts transfected with human IGF-IR cDNA (complete human IGF-IR cDNA: GenBank Acc. No. NM_{—}000875), prepared as described in Kato et al., J. Biol. Chem. 268, 2655-61, 1993, are used (this cell line is hereinafter referred to as “NWT-21” cell line). The cells which overexpress human IGF-IR are cultured in Dulbecco's minimal essential (DMEM) medium, containing 10% Fetal Calf Serum (FCS). For the assay 5,000 cells/well are plated on day 1 on 96-well plates (Costar #3595) in normal growth medium and incubated for 2 days at 37° C. in a standard CO₂ cell incubator. The density of the cells does not exceed 70-80% at day 3. On day 3 the medium is discarded and the cells are incubated for 24 h in minimal medium (DMEM, containing 0.5% FCS). Compounds of formula I [starting from 10 mM dimethyl sulfoxide (DMSO) stock solutions] are added to produce final concentrations of 0.01, 0.03, 0.1, 0.3, 1, 3 and 10 μM to determine the IC₅₀ value. The cells are incubated for 90 min in the presence of a compound of formula I. Thereafter the cells are stimulated with 50 μl IGF-I (final concentration of IGF-I in the well=10 ng/ml; IGF-I is obtained from Sigma; Product Code: I 3769) and incubated for 10 min at 37° C. The medium is discarded and the cells are washed twice with PBS/O (=Phosphate-Buffered Saline without CaCl₂) and lysed for 15 min on ice with 50 μl/well RIPA-buffer [50 mM Tris·HCl, pH=7.2, 120 mM NaCl, 1 mM EDTA, 6 mM EGTA, 1% NP40, 20 mM NaF, 1 mM benzamidine, 15 mM sodium pyrophosphate, 1 mM Phenyl methyl sulphonyl fluoride (PMSF) and 0.5 mM Na₃VO₄] and shaken for 10 min using a 96-well plate shaker (=cellular extracts).

Packard HTRF-96 black plates are coated with 50 μl IGF-IR monoclonal Antibody (mAB) (Santa Cruz; Cat. No.: SC-462) in a concentration of 5 μg/ml at 4° C. overnight. The plates are washed twice with 0.05% (v/v) Tween-20 in Phosphate-Buffered Saline (PBS) and once with nanopure H₂O. Blocking is done for 2 h at room temperature (RT) with 3% Bovine Serum Albumin (BSA) in TBS-T buffer (20 mM Tris·HCl, pH=7.6, 137 mM NaCl, 0.05% Tween-20). After blocking, the plates are washed once with nanopure H₂O.

Cellular extracts (40 μl/well) are pipetted onto the precoated Packard plates, together with 40 μl of the anti-phosphotyrosine mouse mAB PY-20 conjugated with Alkaline Phosphatase (AP) (1:1000 diluted in RIPA buffer; the antibody is obtained from Transduction Labs; Cat. No.: P11120).

After incubating the extracts and the secondary antibody for 2 h at 4° C., the extracts are discarded, the plates are washed twice with 0.05% (v/v) Tween-20 in PBS and once with nanopure water.

90 μl/well AP substrate (CDP-Star; obtained from Tropix; Cat. No.: MS100RY) are then added and the plates are incubated for 45 min at RT in the dark, followed by measuring AP activity in a Packard Top Count Microplate Scintillation Counter. The IC₅₀ values for the compounds of formula I are calculated via linear regression analysis using the GraphPad In-stat program (GraphPad Software, USA). IC₅₀ values in the range of 10 nM to 10 μM, especially in the range of 50 nM to 1 μM are found.

In vivo activity of the compounds of formula I can be shown in the nude mouse xenotransplant model using NWT-21 cells (injected subcutaneously into the flanks of carrier mice) as xenografts and following protocols known in the art. Treatment with the test compound is started as soon as the tumour has reached a mean volume of 100 mm³. Tumour growth is measured two to three times a week and 24 hours after the last treatment by determining the length of two perpendicular axes. The tumour volumes are calculated in accordance with published methods (see Evans et al., Brit. J. Cancer 45, 466-8, 1982). The anti-tumour efficacy is determined as the mean increase in tumour volume of the treated animals divided by the mean increase in tumour volume of the untreated animals (controls) and, after multiplication by 100, is expressed as TIC %. Tumour regression (given in %) is reported as the smallest mean tumour volume in relation to the mean tumour volume at the start of treatment. The test compound is administered daily by gavage.

As an alternative to cell line NWT-21, other cell lines may also be used in the same manner, for example:
the human epithelial cell line A-431; American Type Culture Collection (ATCC), Rockville, Md., USA, Catalogue Number ATCC CRL 1555; cell line from an 85-year-old woman; epidermoid carcinoma cell line;
the MCF-7 breast adenocarcinoma cell line (ATCC No. HTB 22; see also J. Natl. Cancer Inst. (Bethesda) 51, 1409-16, 1973); and
the DU145 prostate carcinoma cell line DU 145 (ATCC No. HTB 81; see also Cancer Res. 37, 4049-58, 1978).

The compounds of formula I exhibit a good pharmacokinetic profile in that for example a good exposure in tumour tissue is observed when a compound of formula I is administered (p.o, i.v. or i.p.) in in vivo efficacy models following protocols well known in the art (e.g. human xenografts in nude mice).

On the basis of these studies, a compound of formula I according to the invention shows therapeutic efficacy especially against proliferative diseases responsive to an inhibition of the IGF-IR tyrosine kinase.

In general, the invention relates also to the use of a compound of formula I for the inhibition of the IGF-IR tyrosine kinase.

In addition to the diseases mentioned above, the compounds of formula I can further be used in the treatment of obesity and are also suitable for the treatment of ischemic retinopathies, such as e.g. diabetic retinopathy and retinopathy of prematurity (ROP) (Smith et al., Nature Medicine 5, 1390-1395, 1999; Hellstrom et al., Proc. Natl. Acad. Sci. USA 98, 5804-5808, 2001). The effectiveness of the compounds of formula I in these diseases can be shown by using in vitro- or in vivo-tests known in the art.

The compounds of formula I can further be used in the treatment of degenerative ocular disorders. Degenerative ocular disorders which may be treated according to this invention include an ocular disease and disorder which may directly or indirectly involve the degeneration of retinal or corneal cells, in particular by apoptosis, including ischemic retinopathies in general, anterior ischemic optic neuropathy, all forms of optic neuritis, age-related macular degeneration (AMD), in its dry forms (dry AMD) and wet forms (wet AMD), diabetic retinopathy, cystoid macular edema (CME), retinal detachment, retinitis pigmentosa, Stargardt's disease, Bests vitelliform retinal degeneration, Leber's congenital amaurosis and other hereditary retinal degenerations, pathologic myopia, neovascular glaucoma, retinopathy of prematurity, and Leber's hereditary optic neuropathy, the after effects of corneal transplantation or of refractive corneal surgery, and herpes keratitis.

Preferably, said ocular disorders are selected from: Dry AMD, wet AMD, diabetic retinopathy, cystoid macular edema (CME), retinal detachment, pathologic myopia, Leber's hereditary optic neuropathy, retinitis pigmentosa, and other hereditary retinal degenerations, and even more preferably, said ocular disorders are selected from: Dry AMD, wet AMD and retinal detachment.

Compounds of formula I are also useful for preventing or combating graft vessel diseases, e.g. allo- or xenotransplant vasculopathies, e.g. graft vessel atherosclerosis or chronic graft rejection, e.g. in a transplant of organ, tissue or cells, e.g. heart, lung, combined heart-lung, liver, kidney or pancreatic transplants (e.g. pancreatic islet cells), or for preventing or treating vein graft stenosis, restenosis and/or vascular occlusion following vascular injury, e.g. caused by catherization procedures or vascular scraping procedures such as percutaneous transluminal angioplasty, laser treatment or other invasive procedures which disrupt the integrity of the vascular intima or endothelium.

With the groups of preferred compounds of formula I mentioned hereinafter, definitions of substituents from the general definitions mentioned hereinbefore may reasonably be used, for example, to replace more general definitions with more specific definitions or especially with definitions characterized as being preferred.

Preference is given to a compound of formual 1, wherein
n is from 0 to 4,
R₁ is hydrogen, unsubstituted or substituted lower alkyl or halogen, R₂ is hydroxy; unsubstituted, mono- or disubstituted amino; a heterocyclic radical containing at least one nitrogen ring atom and being attached to the cyclohexane ring of the molecule of formula I via a nitrogen ring atom; a radical R₅—(C═Y)—NH—, wherein R₅ is unsubstituted or substituted lower alkyl, unsubstituted, mono- or disubstituted amino, a heterocyclic radical, or etherified hydroxy, and Y is oxygen, sulfur or imino; or a radical R₆-sulfonylamino, wherein R₆ is unsubstituted or substituted lower alkyl, unsubstituted, mono- or disubstituted amino or phenyl optionally substituted by lower alkyl, lower alkoxy or nitro,
R₃ is lower alkyl or lower alkoxy, wherein the R₃ substituents can be selected independently of one another if n>1,
R₄ is a radical R₇—CR₈(R₉)—, wherein R₇ is cyclobutyl, cyclopentyl, cyclohexyl, phenyl, furyl, pyrrolyl, thienyl, pyridyl or phenyl substituted by one or more radicals selected from lower alkyl and halogen, and R₈ and R₉ are independently of each other hydrogen, lower alkyl or halogen, and
X is selected from —O—, —NH— and —S—,
or a salt thereof.

Special preference is given to a compound of formula I, wherein
n is 0,
R₁ is hydrogen, unsubstituted or substituted lower alkyl or halogen,
R₂ is hydroxy; unsubstituted, mono- or disubstituted amino; a heterocyclic radical containing at least one nitrogen ring atom and being attached to the cyclohexane ring of the molecule of formula I via a nitrogen ring atom; a radical R—(C═Y)—NH—, wherein R₅ is unsubstituted or substituted lower alkyl, unsubstituted, mono- or disubstituted amino, a heterocyclic radical, or etherified hydroxy, and Y is oxygen, sulfur or imino; or a radical R₆-sulfonylamino, wherein R₆ is unsubstituted or substituted lower alkyl, unsubstituted, mono- or disubstituted amino or phenyl optionally substituted by lower alkyl, lower alkoxy or nitro,
R₄ is benzyl, and
X is selected from —O—, —NH— and —S—,
or a salt thereof.

Preference is especially given to a compound of formula I, wherein
n is 0,
R₁ is hydrogen, unsubstituted or substituted lower alkyl or halogen,
R₂ is hydroxy; unsubstituted, mono- or disubstituted amino; a heterocyclic radical having from 4 to 8 ring members and from 1 to 3 heteroatoms whereby at least one heteroatom is nitrogen and the binding of the heterocyclic radical to the cyclohexane ring of the molecule of formula I occurs via a nitrogen ring atom; a radical R₅—(C═Y)—NH—, wherein R₅ is lower alkyl, unsubstituted, mono- or disubstituted amino, etherified hydroxy, a heterocyclic radical having from 4 to 8 ring members and from 1 to 3 heteroatoms whereby at least one heteroatom is nitrogen and the binding of the heterocyclic radical occurs via a nitrogen ring atom, lower alkyl substituted by said heterocyclic radical or by one or more radicals selected independently of one another from the group consisting of amino, N-lower alkylamino, N,N-di-lower alkylamino, N-lower alkanoylamino, N,N-di-lower alkanoylamino, hydroxy, lower alkoxy, lower alkoxy-lower alkoxy, lower alkanoyl, lower alkanoyloxy, cyano, nitro, carboxy, lower alkoxycarbonyl, carbamoyl, amidino, guanidino, ureido, mercapto, lower alkylthio and halogen, and
Y is oxygen, sulfur or imino; or a radical R₆-sulfonylamino, wherein R₅ is unsubstituted or substituted lower alkyl, unsubstituted, mono- or disubstituted amino or phenyl optionally substituted by lower alkyl, lower alkoxy or nitro,
R₄ is benzyl, and
X is selected from —O—, —NH— and —S—,
or a salt thereof.

Very especially preferred is a compound of formula I, wherein
n is 0,
R₁ is hydrogen, lower alkyl or halogen,
R₂ is hydroxy; unsubstituted, mono- or disubstituted amino; a heterocyclic radical having from 4 to 8 ring members and from 1 to 3 heteroatoms whereby at least one heteroatom is nitrogen and the binding of the heterocyclic radical to the cyclohexane ring of the molecule of formula I occurs via a nitrogen ring atom; a radical R₅—(C═Y)—NH—, wherein R₅ is lower alkyl, unsubstituted or monosubstituted amino, etherified hydroxy, or lower alkyl substituted by a heterocyclic radical having from 4 to 8 ring members and from 1 to 3 heteroatoms whereby at least one heteroatom is nitrogen and the binding of the heterocyclic radical occurs via a nitrogen ring atom, and Y is oxygen or imino; or a radical R₆-sulfonylamino, wherein R₆ is lower alkyl or disubstituted amino,
R₄ is benzyl, and
X is selected from —O—, —NH— and —S—,
or a salt thereof.

Most preferred is a compound of formula I, wherein
n is 0,
R₁ is hydrogen, lower alkyl or halogen,
R₂ is hydroxy, amino, N,N-di-lower alkylamino, pyrimidinyl-amino, 1,4,5,6-tetrahydro-pyrimidinyl-amino, 4,5-dihydro-1H-imidazolyl-amino, azetidin-1-yl, pyrrolidin-1-yl, 1-piperidyl, lower alkyl-piperazin-1-yl, morpholin-4-yl, thiomorpholin-4-yl; a radical R₅—(C═Y)—NH—, wherein R₅ is lower alkyl, lower alkoxy, amino, N-lower alkylamino, N-(phenyl-lower alkyl)-amino, N-(lower alkyl-phenyl-lower alkyl)-amino, N-(lower alkoxy-phenyl-lower alkyl)-amino, N-(morpholin-4-yl-lower alkyl)-amino, N—(N′,N′-di-lower alkylamino-lower alkyl)-amino, lower alkoxy-lower alkoxy, 1-piperidyl-lower alkyl, morpholin-4-yl-lower alkyl or lower alkyl-piperazin-1-yl-lower alkyl, and Y is oxygen or imino; or a radical R₆-sulfonylamino, wherein
R₆ is lower alkyl or N,N-di-lower alkylamino,
R₄ is benzyl, and
X is —O—,
or a salt thereof.

Especially preferred is further a compound of formula I, wherein R₂ is in the 4 position of the cyclohexane ring of the molecule of formula I.

Very special preference is given to a compound of formula I mentioned in the Examples below, or a salt, especially a pharmaceutically acceptable salt, thereof.

Also especially preferred are all compounds of formula I, which in the above-described “Capture ELISA” assay have an IC₅₀ value of less than 1 μM.

The compounds of formula I or salts thereof are prepared in accordance with processes known per se, though not previously described for the manufacture of the compounds of the formula I, especially whereby a) in order to prepare a compound of formula I, in which R₂ is hydroxy, a compound of formula II 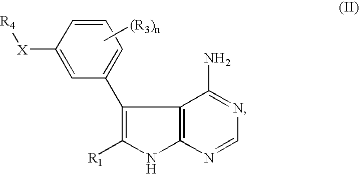
wherein n, R₁, R₃, R₄ and X have the meanings as defined for a compound of formula I, is reacted with methanesulfonic acid hydroxy-cyclohexyl ester;
b) in order to prepare a compound of formula I, in which R₂ is amino, a compound of formula II, wherein n, R₁, R₃, R₄ and X have the meanings as defined for a compound of formula I, is reacted in a first step with a compound of formula III 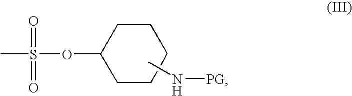
   wherein PG Is an amino protecting group which is removed in a second step;
c) in order to prepare a compound of formula I, in which R₂ is mono- or disubstituted amino or a heterocyclic radical containing at least one nitrogen ring atom and being attached to the cyclohexane ring of the molecule of formula I via a nitrogen ring atom, a compound of formula IV, 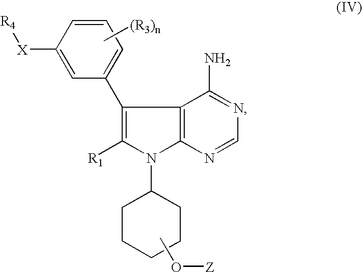
   wherein n, R₁, R₃, R₄ and X have the meanings as defined for a compound of formula I and —O—Z is a leaving group, is reacted with a compound of the formula R₁₀—H in which R₁₀ is mono- or disubsUtuted amino or a heterocyclic radical containing at least one nitrogen ring atom wherein the heterocyclic radical is attached to the hydrogen atom of R₁₀—H via a nitrogen ring atom;
d) in order to prepare a compound of formula I, in which R₂ is a radical R₅—(C═Y)—NH— wherein R₅ is unsubstituted or substituted lower alkyl and Y is oxygen, a compound of formula I, in which R₂ is amino, is reacted with a compound of the formula R₅—(C═O)-Halogen wherein R; is unsubstituted or substituted lower alkyl;
e) in order to prepare a compound of formula I, in which R₂ is a radical R₅—(C═Y)—NH— wherein R₅ is lower alkyl substituted by a heterocyclic radical containing at least one nitrogen ring atom whereby the binding of the heterocyclic radical to lower alkyl occurs via a nitrogen ring atom, and Y is oxygen, a compound of formula V 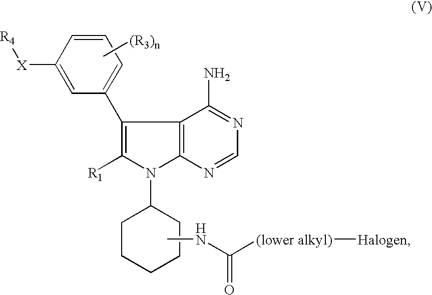
   wherein n, R₁, R₃, R₄ and X have the meanings as defined for a compound of formula I, is reacted with a compound of the formula R₁₁—H in which R₁, is a heterocyclic radical containing at least one nitrogen ring atom wherein the heterocyclic radical is attached to the hydrogen atom of R₁, —H via a nitrogen ring atom;
f) in order to prepare a compound of formula I, in which R₂ is a radical R₅—(C═Y)—NH— wherein —R₅ is unsubstituted, mono- or disubstituted amino or a heterocyclic radical containing at least one nitrogen ring atom whereby the binding of the heterocyclic radical occurs via a nitrogen ring atom and Y is oxygen, a compound of formula I, in which R₂ is a radical R₅—(C═Y)-NH— wherein R₅ is imidazol-1-yl and Y is oxygen, is reacted with a compound of the formula R₅—H, in which R₅ is unsubstituted, mono- or disubstituted amino, or a heterocyclic radical which contains at least one nitrogen ring atom;
g) in order to prepare a compound of formula I, in which R₂ is a radical R₅—(C═Y)-NH— wherein R₅ is unsubstituted or monosubstituted amino and Y is oxygen or sulfur, a compound of formula I, in which R₂ is amino, is reacted with a compound of the formula R₁₂—N═C═Y wherein Y is oxygen or sulfur, the radical R₁₂—NH— corresponding to unsubstituted or monosubstituted amino R₅;
h) in order to prepare a compound of formula I, in which R₂ is a radical R₅—(C═Y)—NH—wherein R₅ is lower alkylamino wherein the lower alkyl moiety is substituted by unsubstituted, mono- or disubstituted amino or by a heterocyclic radical containing at least one nitrogen ring atom whereby the binding of the heterocyclic radical to the lower alkyl moiety occurs via a nitrogen ring atom and Y is oxygen or sulfur, a compound of formula VI 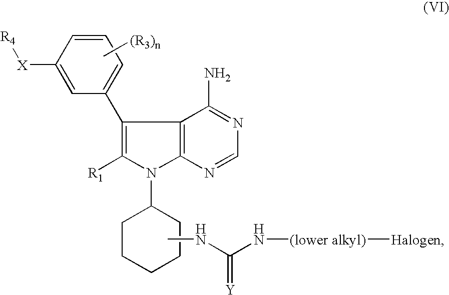
   wherein Y is oxygen or sulfur and n, R₁, R₃, R₄ and X have the meanings as defined for a compound of formula I, is reacted with a compound of the formula R₁₃—H, in which R₁₃ is unsubstituted, mono- or disubstituted amino or a heterocyclic radical containing at least one nitrogen ring atom wherein the heterocyclic radical is attached to the hydrogen atom of R₁₃—H via a nitrogen ring atom;
i) in order to prepare a compound of formula I, in which R₂ is a radical R₅—(C═Y)—NH— wherein R₅ is etherified hydroxy and Y is oxygen, a compound of formula I, in which R₂ is amino, is reacted with a compound of the formula R₅—(C═O)-Halogen wherein R₅ is etherified hydroxy;
i) in order to prepare a compound of formula I, in which R₂ is a radical R₅—(C═Y)—NH— wherein R₅ is lower alkoxy substituted by unsubstituted, mono- or disubstituted amino or by a heterocyclic radical containing at least one nitrogen ring atom whereby the binding of the heterocyclic radical to the lower alkyl moiety of lower alkoxy occurs via a nitrogen ring atom and Y is oxygen, a compound of formula VII 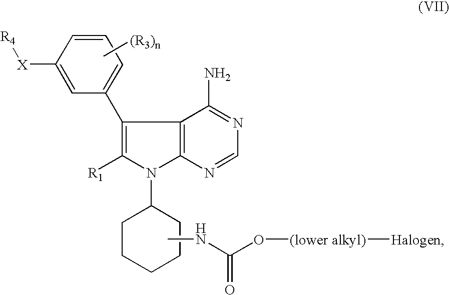
   wherein n, R₁, R₃, R₄ and X have the meanings as defined for a compound of formula I, is reacted with a compound of the formula R₁₄—H, in which R₁₄ is unsubstituted, mono- or disubstituted amino or a heterocyclic radical containing at least one nitrogen ring atom wherein the heterocyclic radical is attached to the hydrogen atom of R₁₄—H via a nitrogen ring atom;
k) in order to prepare a compound of formula I, in which R₂ is a radical R₆-sulfonylamino wherein R₆ has the meanings as defined above under formula I, a compound of formula I, in which R₂ is amino, is reacted with R₆-sulfonyl halide;
l) in order to prepare a compound of formula I, in which R₁ is halogen, a compound of formula I, in which R₁ is hydrogen, is reacted with N-halosuccinimide;
m) in order to prepare a compound of formula I, in which R₁ is lower alkyl, a compound of formula I, in which R₁ is halogen, is reacted with tetra(lower alkyl) tin;
n) in order to prepare a compound of formula I, a compound of formula II, wherein n, R₁, R₃, R₄ and X have the meanings as defined for a compound of formula I, is reacted with a compound of formula VIII 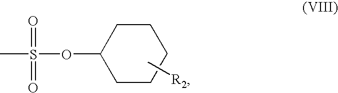
   wherein R₂ has the meanings as defined for a compound of formula I;
wherein functional groups which are present in the starting compounds of processes a) to n) and are not intended to take part in the reaction, are present in protected form if necessary, and protecting groups that are present are cleaved, wherein said starting compounds may also exist in the form of salts provided that a salt-forming group is present and a reaction in salt form is possible;
and, if so desired, a compound of formula I thus obtained is converted into another compound of formula I, a free compound of formula I is converted into a salt, an obtained salt of a compound of formula I is converted into the free compound or another salt, and/or a mixture of isomeric compounds of formula I is separated into the individual isomers.
   Description of the Process Variants:
   Regarding Process a):

The reaction between a compound of formula II and methanesulfonic acid hydroxy-cyclohexyl ester preferably takes place in the presence of a suitable base, such as potassium carbonate, and in the presence of 18-crown-6 ether, in a suitable inert solvent, such as for example N,N-dimethylformamide, preferably at elevated temperature such as around 70-80° C. Methanesulfonic acid hydroxy-cyclohexyl ester is especially methanesulfonic acid 4-hydroxy-cyclohexyl ester.

Regarding Process b):

The reaction between a compound of formula II and a compound of formula III preferably takes place under the reaction conditions described above for process a). The amino protecting group PG is preferably tert-butoxycarbonyl which can be removed in the presence of an acid such as especially formic acid, preferably at elevated temperature such as around 50° C.

Regarding Process c):

The reaction between a compound of formula IV and a compound of the formula R₁₀—H preferably takes place at at elevated temperature such as around 70° C. If at the reaction temperature the compound of formula R₁₀—H is in the form of a liquid and the compound of formula IV is soluble therein, no additional solvent is needed. The leaving group —O—Z is one known in the art, preferably p-toluenesulfonyloxy.

Regarding Process d):

The reaction between a compound of formula I, in which R₂ is amino, and a compound of the formula R₅—(C═O)-Halogen, wherein R₅ is unsubstituted or substituted lower alkyl and Halogen is preferably chlorine, preferably takes place in a suitable inert solvent, such as for example N,N-dimethylformamide, preferably at RT.

Regarding Process e):

The reaction between a compound of formula V and a compound of the formula R₁₁-H preferably takes place in a suitable inert solvent, especially alcohols, e.g. lower alcohols, such as ethanol, preferably at the reflux temperature of the solvent employed. In a compound of formula V, Halogen is preferably chlorine.

Regarding Process f):

The reaction between a compound of formula I, in which R₂ is a radical R₅—(C═Y)—NH— wherein R₅ is imidazol-1-yl, and Y is oxygen, and a compound of the formula R₅—H, in which R₅ is unsubstituted, mono- or disubstituted amino, or a heterocyclic radical which contains at least one nitrogen ring atom, preferably takes place in the presence of triethylamine, in a suitable inert solvent, such as for example acetonitrile, and in an inert, for example an argon, atmosphere, preferably at RT.

Regarding Process g):

The reaction between a compound of formula I, in which R₂ is amino, and a compound of the formula R₁₂—N═C═Y preferably takes place in a suitable inert solvent, such as for example acetonitrile, preferably at RT.

Regarding Process h):

The reaction between a compound of formula VI and a compound of the formula R₁₃—H preferably takes place in a suitable inert solvent, especially alcohols, e.g. lower alcohols, such as ethanol, preferably at the reflux temperature of the solvent employed. In a compound of formula VI, Halogen is preferably chlorine or bromine.

Regarding Process i):

The reaction between a compound of formula I, in which R₂ is amino, and a compound of the formula R₅—(C═O)-Halogen, wherein R₅ is etherified hydroxy and Halogen is preferably chlorine, preferably takes place in the presence of triethylamine, in a suitable inert solvent, such as for example dichloromethane, preferably at RT.

Regarding Process j):

The reaction between a compound of formula VII and a compound of the formula R₁₄—H preferably takes place in a suitable inert solvent, such as for example acetonitrile, preferably at the reflux temperature of the solvent employed. In a compound of formula VII, Halogen is preferably bromine.

Regarding Process k):

The reaction between a compound of formula I, in which R₂ is amino, and R₆-sulfonyl halide, in which R₆ is as defined above under formula I, preferably takes place in the presence of triethylamine, in a suitable inert solvent, such as for example dichloromethane, and in an inert, for example an argon, atmosphere, preferably at RT. In R₆-sulfonyl halide, halide is preferably chloride.

Regarding Process l):

The reaction between a compound of formula I, in which R₁ is hydrogen, and N-halosuccinimide, preferably takes place in a suitable inert solvent, such as for example N,N-dimethylformamide, and in an inert, for example an argon, atmosphere, preferably at RT in the dark. N-halosuccinimide is preferably N-bromosuccinimide.

Regarding Process m):

The reaction between a compound of formula I, in which R₁ is halogen, and tetra(lower alkyl) tin, preferably takes place in the presence of tetrakis(triphenylphosphine) palladium (0), in a suitable inert solvent, such as for example N,N-dimethylformamide, and in an inert, for example an argon, atmosphere, preferably at elevated temperature such as around 100° C. Tetra(lower alkyl) tin is preferably tin tetramethyl.

Regarding Process n):

The reaction between a compound of formula II and a compound of formula VIII preferably takes place in the presence of a suitable base, such as potassium carbonate, and in the presence of 18-crown-6 ether, in a suitable inert solvent, such as for example N,N-dimethylformamide, preferably at elevated temperature such as around 70-80° C.

Additional Process Steps

In the additional process steps, carried out as desired, functional groups of the starting compounds which should not take part in the reaction may be present in unprotected form or may be protected for example by one or more protecting groups. The protecting groups are then wholly or partly removed according to one of the known methods. Protecting groups, and the manner in which they are introduced and removed are described, for example, in “Protective Groups in Organic Chemistry”, Plenum Press, London, New York 1973, and in “Methoden der organischen Chemie”, Houben-Weyl, 4th edition, Vol. 15/1, Georg-Thieme-Verlag, Stuttgart 1974 and in Theodora W. Greene, “Protective Groups in Organic Synthesis”, John Wiley & Sons, New York 1981. A characteristic of protecting groups is that they can be removed readily, i.e. without the occurrence of undesired secondary reactions, for example by solvolysis, reduction, photolysis or alternatively under physiological conditions.

The end products of formula I may however also contain substituents that can also be used as protecting groups in starting materials for the preparation of other end products of formula I. Thus, within the scope of this text, only a readily removable group that is not a constituent of the particular desired end product of formula I is designated a “protecting group”, unless the context indicates otherwise.

A compound of formula I can be converted to a corresponding N-oxide. The reaction is carried out with a suitable oxidizing agent, preferably a peroxide, for example m-chloroperbenzoic acid, in a suitable solvent, e.g. halogenated hydrocarbon, typically chloroform or dichloromethane, or in a lower alkanecarboxylic acid, typically acetic acid, preferably at a temperature between 0° C. and the boiling temperature of the reaction mixture, especially at about RT.

General Process Conditions

All process steps described here can be carried out under known reaction conditions, preferably under those specifically mentioned, in the absence of or usually in the presence of solvents or diluents, preferably those that are inert to the reagents used and able to dissolve them, in the absence or presence of catalysts, condensing agents or neutralising agents, for example ion exchangers, typically cation exchangers, for example in the H⁺ form, depending on the type of reaction and/or reactants at reduced, normal, or elevated temperature, for example in the range from −100° C. to about 190° C., preferably from about −80° C. to about 150° C., for example at −80 to 60° C., at RT, at −20 to 40° C. or at the boiling point of the solvent used, under atmospheric pressure or in a closed vessel, if need be under pressure, and/or in an inert, for example an argon or nitrogen, atmosphere.

The invention relates also to those embodiments of the process in which one starts from a compound obtainable at any stage as an intermediate and carries out the missing steps, or breaks off the process at any stage, or forms a starting material under the reaction conditions, or uses said starting material in the form of a reactive derivative or salt, or produces a compound obtainable by means of the process according to the invention under those process conditions, and further processes the said compound in situ. In the preferred embodiment, one starts from those starting materials which lead to the compounds described hereinabove as preferred.

In the preferred embodiment, a compound of formula I (or an N-oxide thereof) is prepared according to the processes and process steps defined in the Examples.

The compounds of formula I (or N-oxides thereof, including their salts, are also obtainable in the form of hydrates, or their crystals can include for example the solvent used for crystallisation (present as solvates).

Starting Materials

New starting materials and/or intermediates, as well as processes for the preparation thereof, are likewise the subject of this invention. In the preferred embodiment, such starting materials are used and reaction conditions so selected as to enable the preferred compounds to be obtained.

The starting materials used in the above described processes a) to n) are known, capable of being prepared according to known processes (see also WO 97/28161), or commercially obtainable; in particular, they can be prepared using processes as described in the Examples.

In the preparation of starting materials, existing functional groups which do not participate in the reaction should, if necessary, be protected. Preferred protecting groups, their introduction and their removal are described above or in the examples. In place of the respective starting materials and transients, salts thereof may also be used for the reaction, provided that salt-forming groups are present and the reaction with a salt is also possible. Where the term starting materials is used hereinbefore and hereinafter, the salts thereof are always included, insofar as reasonable and possible.

A compound of formula II can be prepared for example analogously as described for compounds of formula IV in WO 97/28161.

A compound of formula IV can be prepared for example by transforming the R₂ hydroxy group of a compound of formula I, in which R₂ is hydroxy, into a leaving group —O—Z according to procedures known in the art. A compound of formula IV, in which Z is p-toluenesulfonyl, can be prepared for example by reacting a compound of formula I, in which R₂ is hydroxy, with p-toluenesulfonyl halide, preferably p-toluenesulfonyl chloride, in an inert solvent, for example pyridine, preferably at −10° C.

A compound of formula V can be prepared for example by reacting a compound of formula I, in which R₂ is amino, with halogen-lower alkylcarbonyl halide, preferably chloro-lower alkylcarbonyl chloride, in the presence of triethylamine, in an inert solvent, for example acetonitrile, preferably at RT.

A compound of formula I, in which R₂ is a radical R₅—(C═Y)—NH— wherein R₅ is imidazol-1-yl and Y is oxygen, can be obtained for example by reacting a compound of formula I, in which R₂ is amino, with 1,1-carbonyldiimidazole, in the presence of triethylamine, in an inert solvent, for example acetonitrile, and in an inert, for example an argon, atmosphere, preferably at RT.

A compound of formula VI can be obtained for example by reacting a compound of formula I, in which R₂ is amino, with a compound of the formula halogen-lower alkyl-N═C═Y, wherein Y is oxygen or sulfur and halogen is preferably chlorine and bromine, in an inert solvent, for example acetonitrile, preferably at RT.

A compound of formula VII can be prepared for example by reacting a compound of formula I, in which R₂ is amino, with halogen-lower alkyl halogen formate, preferably bromo-lower alkyl chloroformate, in the presence of triethylamine, in an inert solvent, for example dichloromethane, preferably at RT.

A compound of formula I, in which R₁ is hydrogen, can be obtained according to processes a)-k) or n).

The remaining starting materials are known, capable of being prepared according to known processes, or commercially available; or in particular, they can be prepared using processes as described in the Examples.

Pharmaceutical Compositions, Methods, Uses and Combinations

The present invention relates also to pharmaceutical compositions that comprise a compound of formula I, or a pharmaceutically acceptable salt thereof, as active ingredient and that can be used especially in the treatment of the diseases mentioned at the beginning.

The present invention also relates to pro-drugs of a compound of formula I that convert in vivo to the compound of formula I as such. Any reference to a compound of formula I is therefore to be understood as referring also to the corresponding pro-drugs of the compound of formula I, as appropriate and expedient.

Compositions for enteral administration, such as nasal, buccal, rectal or, especially, oral administration, and for parenteral administration, such as intravenous, intramuscular or subcutaneous administration, to warm-blooded animals, especially humans, are especially preferred. The compositions contain the active ingredient alone or, preferably, together with a pharmaceutically acceptable carrier. The dosage of the active ingredient depends upon the disease to be treated and upon the species, its age, weight, and individual condition, the individual pharmacokinetic data, and the mode of administration.

The invention relates also to compounds of formula I, or a pharmaceutically acceptable salt thereof, as such or in the form of a pharmaceutical composition, for use in a method for the prophylactic or especially therapeutic treatment of the human or animal body, to a process for the preparation thereof (especially in the form of compositions for the treatment of tumours) and to a method of treating the above-mentioned diseases, primarily tumour diseases, especially those mentioned above.

The invention relates also to processes and to the use of compounds of formula I, or a pharmaceutically acceptable salt thereof, for the preparation of pharmaceutical compositions which comprise compounds of formula I, or a pharmaceutically acceptable salt thereof, as active component (active ingredient).

If desired, the said pharmaceutical compositions may also contain further active components, such as other chemotherapy drugs, and/or may be used in combination with known therapeutic processes, for example the administration of hormonal medicines or radiation.

Preference is for a pharmaceutical composition which is suitable for administration to a warm-blooded animal, especially humans or commercially useful mammals suffering from a disease which responds to an inhibition of the IGF-IR tyrosine kinase or of the IGF-IR-dependent cell proliferation, especially a neoplastic disease, comprising an effective quantity of a compound of formula I for the inhibition of the IGF-IR tyrosine kinase or of the IGF-IR-dependent cell proliferation, or a pharmaceutically acceptable salt thereof, together with at least one pharmaceutically acceptable carrier.

A pharmaceutical composition for the prophylactic or especially therapeutic management of neoplastic and other proliferative diseases of a warm-blooded animal, especially a human or a commercially useful mammal requiring such treatment, especially suffering from such a disease, comprising as active ingredient in a quantity that is prophylactically or especially therapeutically active against said diseases a new compound of formula I, or a pharmaceutically acceptable salt thereof, is likewise preferred.

The pharmaceutical compositions comprise from approximately 1% to approximately 95% active ingredient, single-dose administration forms comprising in the preferred embodiment from approximately 20% to approximately 90% active ingredient and forms that are not of single-dose type comprising in the preferred embodiment from approximately 5% to approximately 20% active ingredient. Unit dose forms are, for example, coated and uncoated tablets, ampoules, vials, suppositories or capsules. Examples are capsules containing from about 0.05 g to about 1.0 g of active substance.

The pharmaceutical compositions of the present invention are prepared in a manner known per se, for example by means of conventional mixing, granulating, coating, dissolving or ly-ophilising processes.

The invention relates likewise to a process or a method for the treatment of one of the pathological conditions mentioned hereinabove, especially a disease which responds to an inhibition of the IGF-IR tyrosine kinase or of the IGF-IR-dependent cell proliferation, especially a corresponding neoplastic disease. The compounds of formula I, or a pharmaceutically acceptable salt thereof, can be administered as such or in the form of pharmaceutical compositions, prophylactically or therapeutically, preferably in an amount effective against the said diseases, to a warm-blooded animal, for example a human, requiring such treatment, the compounds especially being used in the form of pharmaceutical compositions. In the case of an individual having a bodyweight of about 70 kg the daily dose administered is from approximately 0.1 g to approximately 5 g, preferably from approximately 0.5 g to approximately 2 g, of a compound of the present invention.

The present invention relates especially also to the use of a compound of formula I, or a pharmaceutically acceptable salt thereof, especially a compound of formula I which is said to be preferred, or a pharmaceutically acceptable salt thereof, as such or in the form of a pharmaceutical composition with at least one pharmaceutically acceptable carrier, for the therapeutic and also prophylactic management of one or more of the diseases mentioned hereinabove, preferably a disease which responds to an inhibition of the IGF-IR tyrosine kinase or of the IGF-IR-dependent cell proliferation, especially a neoplastic disease, in particular if the said disease responds to an inhibition of the IGF-IR tyrosine kinase or of the IGF-IR-dependent cell proliferation.

The present invention relates especially also to the use of a compound of formula I, or a pharmaceutically acceptable salt thereof, especially a compound of formula I which is said to be preferred, or a pharmaceutically acceptable salt thereof, for the preparation of a pharmaceutical composition for the therapeutic and also prophylactic management of one or more of the diseases mentioned hereinabove, especially a neoplastic disease, in particular if the disease responds to an inhibition of the IGF-IR tyrosine kinase or of the IGF-IR-dependent cell proliferation. A compound of formula I may also be used to advantage in combination with other antiproliferative agents. Such antiproliferative agents include, but are not limited to aromatase inhibitors, antiestrogens, topoisomerase I inhibitors, topoisomerase II inhibitors, microtubule active agents, alkylating agents, histone deacetylase inhibitors, proteasome inhibitors, farnesyl transferase inhibitors, COX-2 inhibitors, MMP inhibitors, mTOR inhibitors, antineoplastic antimetabolites, platin compounds, compounds decreasing the protein kinase activity and further anti-angiogenic compounds, gonadorelin agonists, anti-androgens, bengamides, bisphosphonates, antiproliferative antibodies, antiproliferative proteins, anthracyclines and dexamethasone (Decadron®).

The term “aromatase inhibitors” as used herein relates to compounds which inhibit the estrogen production, i.e. the conversion of the substrates androstenedione and testosterone to estrone and estradiol, respectively. The term includes, but is not limited to steroids, especially exemestane and formestane and, in particular, non-steroids, especially aminoglutethimide, vorozole, fadrozole, anastrozole and, very especially, letrozole. Exemestane can be administered, e.g., in the form as it is marketed, e.g. under the trademark AROMASIN™. Formestane can be administered, e.g., in the form as it is marketed, e.g. under the trademark LENTARON™. Fadrozole can be administered, e.g., in the form as it is marketed, e.g. under the trademark AFEMA™. Anastrozole can be administered, e.g., in the form as it is marketed, e.g. under the trademark ARIMIDEX™. Letrozole can be administered, e.g., in the form as it is marketed, e.g. under the trademark FEMARA™ or FEMAR™. Aminoglutethimide can be administered, e.g., in the form as it is marketed, e.g. under the trademark ORIMETEN™.

A combination of the invention comprising an antineoplastic agent which is an aromatase inhibitor is particularly useful for the treatment of hormone receptor positive breast tumors.

The term “antiestrogens” as used herein relates to compounds which antagonize the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX™. Raloxifene hydrochloride can be administered, e.g., in the form as it is marketed, e.g. under the trademark EVISTA™. Fulvestrant can be formulated as disclosed in U.S. Pat. No. 4,659,516 or it can be administered, e.g., in the form as it is marketed, e.g. under the trademark FASLODEX™.

The term “topoisomerase I inhibitors” as used herein includes, but is not limited to topotecan, irinotecan, 9-nitrocamptothecin and the macromolecular camptothecin conjugate PNU-166148 (compound A1 in WO99/17804). Irinotecan can be administered, e.g., in the form as it is marketed, e.g. under the trademark CAMPTOSAR™. Topotecan can be administered, e.g., in the form as it is marketed, e.g. under the trademark HYCAMTIN™.

The term “topoisomerase II inhibitors” as used herein includes, but is not limited to the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide. Etoposide can be administered, e.g., in the form as it is marketed, e.g. under the trademark ETOPOPHOS™. Teniposide can be administered, e.g., in the form as it is marketed, e.g. under the trademark VM 26-BRISTOL™. Doxorubicin can be administered, e.g., in the form as it is marketed, e.g. under the trademark ADRIBLASTIN™. Epirubicin can be administered, e.g., in the form as it is marketed, e.g. under the trademark FARMORUBICIN™. Idarubicin can be administered, e.g., in the form as it is marketed, e.g. under the trademark ZAVEDOS™. Mitoxantrone can be administered, e.g., in the form as it is marketed, e.g. under the trademark NOVANTRON™.

The term “microtubule active agents” relates to microtubule stabilizing and microtubule destabilizing agents including, but not limited to the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D. Docetaxel can be administered, e.g., in the form as it is marketed, e.g. under the trademark TAXOTERE™. Vinblastine sulfate can be administered, e.g., in the form as it is marketed, e.g. under the trademark VINBLASTIN R.P.™. Vincristine sulfate can be administered, e.g., in the form as it is marketed, e.g. under the trademark FARMISTIN™. Discodermolide can be obtained, e.g., as disclosed in U.S. Pat. No. 5,010,099.

The term “alkylating agents” as used herein includes, but is not limited to cyclophosphamide, ifosfamide, melphalan and temozolomide. Cyclophosphamide can be administered, e.g., in the form as it is marketed, e.g. under the trademark CYCLOSTIN™. Ifosfamide can be administered, e.g., in the form as it is marketed, e.g. under the trademark HOLOXAN™. Temozolomide can be administered, e.g., in the form as it is marketed, e.g. under the trademark TEMODAL®.

The term “histone deacetylase inhibitors” relates to compounds which inhibit the histone deacetylase and which possess antiproliferative activity. Such compounds include e.g. LAQ824, MS-275, SAHA, FK228, Trichostatin A and CI-994.

The term “proteasome inhibitors” relates to compounds which inhibit the proteasome and which possess antiproliferative activity, such as e.g. the compound PS-341.

The term “farnesyl transferase inhibitors” relates to compounds which inhibit the farnesyl transferase and which possess antiproliferative activity.

The term “COX-2 inhibitors” relates to compounds which inhibit the cyclooxygenase type 2 enyzme (COX-2) and which possess antiproliferative activity such as celecoxib (Celebrex®), rofecoxib (Vioxx®) and lumiracoxib (COX189).

The term “MMP inhibitors” relates to compounds which inhibit the matrix metalloproteinase (MMP) and which possess antiproliferative activity.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “antineoplastic antimetabolites” includes, but is not limited to 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA™), LY264618 (LOMOTREXOL™) and OGT719.

The term “platin compounds” as used herein includes, but is not limited to carboplatin, cisplatin and oxaliplatn. Carboplatin can be administered, e.g., in the form as it is marketed, e.g. under the trademark CARBOPLAT™. Oxaliplatin can be administered, e.g., in the form as it is marketed, e.g. under the trademark ELOXATIN™.

The term “compounds decreasing the protein kinase activity and further anti-angiogenic compounds” as used herein includes, but is not limited to compounds which decrease the activity of e.g. the Vascular Endothelial Growth Factor (VEGF), the Epidermal Growth Factor (EGF), c-Src, protein kinase C, protein kinase B, Platelet-derived Growth Factor (PDGF), Bcr-Abl tyrosine kinase, c-kit, Flt-3 and Cyclin-dependent kinases (CDKs), and anti-angiogenic compounds having another mechanism of action than decreasing the protein kinase activity.

Compounds which decrease the activity of VEGF are especially compounds which inhibit the VEGF receptor, especially the tyrosine kinase activity of the VEGF receptor, and compounds binding to VEGF, and are in particular those compounds, proteins and monoclonal antibodies generically and specifically disclosed in WO 98/35958 (describing compounds of formula I), WO 00/09495, WO 00/27820, WO 00/59509, WO 98/11223, WO 00/27819, WO 01/55114, WO 01/58899 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, vol. 27, no. 1, pp 14-21, 1999; in WO 00/37502 and WO 94/10202; Angiostatin™, described by M. S. O'Reilly et al, Cell 79, 1994, 315-328; and Endostatin™, described by M. S. O'Reilly et al, Cell 88, 1997, 277-285; compounds which decrease the activity of EGF are especially compounds which inhibit the EGF receptor, especially the tyrosine kinase activity of the EGF receptor, and compounds binding to EGF, and are in particular those compounds generically and specifically disclosed in WO 97/02266 (describing compounds of formula IV), EP 0 564 409, WO 99/03854, EP 0520722, EP 0 566 226, EP 0 787 722, EP 0 837 063, WO 98/10767, WO 97/30034, WO 97/49688, WO 97/38983 and, especially, WO 96/33980;
compounds which decrease the activity of c-Src include, but are not limited to, compounds inhibiting the c-Src protein tyrosine kinase activity as defined below and to SH2 interaction inhibitors such as those disclosed in WO97/07131 and WO97/08193;
compounds inhibiting the c-Src protein tyrosine kinase activity include, but are not limited to, compounds belonging to the structure classes of pyrrolopyrimidines, especially pyrrolo[2,3-d]pyrimidines, purines, pyrazopyrimidines, especially pyrazo[3,4-d]pyrimidines, pyrazopyrimidines, especially pyrazo[3,4-d]pyrimidines and pyridopyrimidines, especially pyrido[2,3-d]pyrimidines. Preferably, the term relates to those compounds disclosed in WO 96/10028, WO 97/28161, WO97/32879 and WO97/49706;
compounds which decreases the activity of the protein kinase C are especially those staurosporine derivatives disclosed in EP 0 296 110 (pharmaceutical preparation described in WO 00/48571) which compounds are protein kinase C inhibitors;
further specific compounds that decrease protein kinase activity and which may also be used in combination with the compounds of the present invention are Imatinib (Gleevec®/Glivec®), PKC412, Iressa™ (ZD1839), PKI166, PTK787, ZD6474, GW2016, CHIR-200131, CEP-7055/CEP-5214, CP-547632 and KRN-633;
anti-angiogenic compounds having another mechanism of action than decreasing the protein kinase activity include, but are not limited to e.g. thalidomide (THALOMID), celecoxib (Celebrex) and ZD6126.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX™. Abarelix can be formulated, eg. as disclosed in U.S. Pat. No. 5,843,901.

The term “anti-androgens” as used herein includes, but is not limited to bicalutamide (CASODEX™), which can be formulated, e.g. as disclosed in U.S. Pat. No. 4,636,505.

The term “bengamides” relates to bengamides and derivatives thereof having aniproliferative properties.

The term “bisphosphonates” as used herein includes, but is not limited to etridonic acid, clodronic acid, tiludronic acid, pamidronic acid, alendronic acid, ibandronic acid, risedronic acid and zoledronic acid. “Etridonic acid” can be administered, e.g., in the form as it is marketed, e.g. under the trademark DIDRONEL™. “Clodronic acid” can be administered, e.g., in the form as it is marketed, e.g. under the trademark BONEFOS™. “Tiludronic acid” can be administered, e.g., in the form as it is marketed, e.g. under the trademark SKELID™. “Pamidronic acid” can be administered, e.g., in the form as it is marketed, e.g. under the trademark AREDIA™. “Alendronic acid” can be administered, e.g., in the form as it is marketed, e.g. under the trademark FOSAMAX™. “Ibandronic acid” can be administered, e.g., in the form as it is marketed, e.g. under the trademark BONDRANAT™. “Risedronic acid” can be administered, e.g., in the form as it is marketed, e.g. under the trademark ACTONEL™. “Zoledronic acid” can be administered, e.g., in the form as it is marketed, e.g. under the trademark ZOMETA™.

The term “antiproliferative antibodies” as used herein includes, but is not limited to trastuzumab (Herceptin™), Trastuzumab-DM1, erlotinib (Tarceva™), bevacizumab (Avastin™), rituximab (Rituxan®), PRO64553 (anti-CD40) and 2C4 Antibody.

The term “anthracyclines” includes, but is not limited to Adriamycin, Daunomycin, Idarubicin and Mitoxantrone.

The term “antiproliferative proteins” includes e.g. TRAIL/Apo2L.

The structure of the active agents identified by code nos., generic or trade names may be taken from the actual edition of the standard compendium “The Merck Index” or from data-bases, e.g. Patents International (e.g. IMS World Publications).

The above-mentioned compounds, which can be used in combination with a compound of formula I, can be prepared and administered as described in the art such as in the documents cited above.

### EXAMPLES

The following Examples serve to illustrate the invention without limiting its scope.

Temperatures are measured in degrees Celsius. Unless otherwise indicated, the reactions take place at room temperature.

The R_{f} values which indicate the ratio of the distance moved by each substance to the distance moved by the eluent front are determined on silica gel thin-layer plates (Merck, Darmstadt, Germany) by thin-layer chromatography using the respective named solvent systems.

The short forms and abbreviations used have the following definitions:
ES-MS electron spray-mass spectroscopy
h hour(s)
Me methyl
min minute(s)
RT room temperature
TFA trifluoroacetic acid
t_{R} retention times
v volume
   Analytical HPLC Conditions:
   Gradient 1 (“Grad 1”):

Linear gradient over 7 min of MeCN/0.09% TFA and H₂O/0.1% TFA from 1:49 to 1:0 and 3 min at 1:0, detection at 215 nm, flow rate 2.0 m/min. Column: Nucleosil C18-column (250×4.6 mm, 5 μm, 100 Å).

Gradient 2 (“Grad 2”):

Linear gradient over 10 min of MeCN/0.09% TFA and H₂O/0.1% TFA from 1:49 to 3:2, detection at 215 nm, flow rate 2.0 m/min. Column: Nucleosil C18-column (250×4.6 mm, 5 μm, 100 Å).

### Example 1A

### cis-4-[4-Amino-5-(3-benzyloxy-phenyl)-pyrrolo[2,3-d]pyrimidin-7-yl]-cyclohexanol; and

### Example 1B

### trans-4-[4-Amino-5-(3-benzyloxy-phenyl)-pyrrolo[2,3-d]pyrimidin-7-yl]-cyclohexanol

### Step 1.1: Methanesulfonic acid 4-hydroxy-cyclohexyl ester

20 g of cyclohexane-1,4-diol (Fluka, Buchs, Switzerland) are dissolved in 200 ml of pyridine at 0° C. and 13.4 ml of methanesulfonyl chloride are added thereto in small portions over 5 h. After stirring for 16 h at RT, working-up is effected by partitioning between water and dichloromethane. The organic layer is concentrated in vacuo, and the crude product is purified by flash chromatograpy (dichloromethane/methanol, 49:1, v/v) to yield the title compound. R_{f} 0.36 (chloroform/methanol/water/acetic acid, 900:100:10:5, v/v/v/v).

### Step 1.2: cis- and trans-4-[4-Amino-5-(3-benzyloxy-phenyl)-pyrrolo[2,3-d]pyrimidin-7-yl]-cyclohexanol

A mixture of 5.3 g of 5-(3-benzyloxy-phenyl)-7H-pyrrolo[2,3-d]pyrimidin-4-ylamine, which is obtained as described in Example 5 of WO 97/28161, 4.6 g of powdered potassium carbonate and 8.8 g of 18-crown-6-ether in 70 ml of dimethylformamide is stirred for 20 min at 70° C. 3.9 g of methanesulfonic acid 4-hydroxy-cyclohexyl ester is added. After stirring for 16 h at 70° C., additional 5-(3-benzyloxy-phenyl)-7H-pyrrolo[2,3-d]pyrimidin-4-ylamine (0.53 g), potassium carbonate (0.46 g) and 18-crown-6-ether (0.88 g) are added, and the mixture is stirred again for 24 h at 70° C. Working-up is effected by partitioning between water and ethyl acetate. The organic layer is dried over magnesium sulfate and concentrated in vacuo. The crude product is purified by flash column chromatography (dichloromethane/methanol, 92:8, v/v) and medium-pressure liquid chromatography (Merck, LICHROPREP RP-18, 15-25 μm bead diameter, reversed phase column material based on C₁₈-derivatised silicagel, Merck, Darmstadt, FRG; the chromatography is performed using an acetonitrile-water gradient containing 0.1% trifluoroacetic acid) yielding the pure cis- and trans-structural isomers of the title compound.
cis-4-[4-Amino-5-(3-benzyloxy-phenyl)-pyrrolo[2,3-d]pyrimidin-7-yl]-cyclohexanol

Analytical HPLC: t_{R}=6.84 min (Grad 1); ES-MS: m/eₒ=415.3.
trans-4-[4-Amino-5-(3-benzyloxy-phenyl)-pyrrolo[2,3-d]pyrimidin-7-yl]-cyclohexanol

Analytical HPLC: t_{R}=6.74 min (Grad 1); ES-MS: m/e₀=415.3.

### Example 2

### cis-5-(3-Benzyloxy-phenyl)-7-(4-piperidin-1-yl-cyclohexyl)-7H-pyrrolo[2,3-d]pyrimidin-4-ylamine

### Step 2.1: trans-Toluene-4-sulfonic acid 4-[4-amino-5-(3-benzyloxy-phenyl)-pyrrolo[2,3-d]pyrimidin-7-yl]-cyclohexyl ester

300 mg of trans-4-[4-amino-5-(3-benzyloxy-phenyl)-pyrrolo[2,3-d]pyrimidin-7-yl]-cyclohexanol (Example 1B) are dissolved in 5 ml of dichloromethane at −10° C. 414 mg of p-toluenesulfonyl chloride are added under argon and the solution is stirred for 48 h at −10° C. After this time, the soution is evaporated to dryness and the working-up is effected by partitioning between water and ethyl acetate. The organic layer is concentrated in vacuo. Purification of the crude product by flash column chromatography (dichloromethane/acetonitrile, 1:1, v/v) yields the title compound.

Analytical HPLC: t_{R}=8.36 min (Grad 1); ES-MS: m/eₒ=569.1.

### Step 2.2: cis-5-(3-Benzyloxy-phenyl)-7-(4-piperidin-1-yl-cyclohexyl)-7H-pyrrolo[2,3-d]pyrimidin-4-ylamine

33 mg of trans-toluene-4-sulfonic acid 4-[4-amino-5-(3-benzyloxy-phenyl)-pyrrolo[2,3-d]pyrimidin-7-yl]-cyclohexyl ester are dissoved in 4 ml of acetonitrile/ethyl acetate (3:1, v/v) and 2.1 ml of piperidine are added. The solution is stirred at 70° C. for 72 h. After this time, the solution is evaporated to dryness and the crude compound is purified by medium-pressure liquid chromotagraphy.

Analytical HPLC: t_{R}=6.31 min (Grad 1); ES-MS: m/eₒ=482.3.

### Example 3

### trans-5-(3-Benzyloxy-phenyl)-7-(4-piperidin-1-yl-cyclohexyl)-7H-pyrrolo[2,3-d]pyrimidin-4-ylamine

The title compound is obtained as described in Example 2 using cis-4-[4-amino-5-(3-benzyloxy-phenyl)-pyrrolo[2,3-d]pyrimidin-7-yl]-cyclohexanol (Example 1A) as starting material.

Analytical HPLC: t_{R}=6.30 min (Grad 1); ES-MS: m/e₀=482.2.

### Example 4

### cis-5-(3-Benzyloxy-phenyl)-7-(4-pyrrolidin-1-yl-cyclohexyl)-7H-pyrrolo[2,3-d]pyrimidin-4-ylamine

The title compound is obtained as described in Example 2 using pyrrolidine.

Analytical HPLC: t_{R}=6.78 min (Grad 1); ES-MS: m/eₒ, =468.3.

### Example 5

### trans-5-(3-Benzyloxy-phenyl)-7-(4-pyrrolidin-1-yl-cyclohexyl)-7H-pyrrolo[2,3-d]pyrimidin-4-ylamine

The title compound is obtained as described in Example 2 using cis-4-[4-amino-5-(3-benzyloxy-phenyl)-pyrrolo[2,3-d]pyrimidin-7-yl]-cyclohexanol (Example 1A) as starting material and pyrrolidine.

Analytical HPLC: t_{R}=6.23 min (Grad 1); ES-MS: m/eₒ=468.3.

### Example 6

### cis-5-(3-Benzyloxy-phenyl)-7-[4-(4-methyl-piperazin-1-yl)-cyclohexyl]-7H-pyrrolo[2,3-d]pyrimidin-4-ylamine

The title compound is obtained as described in Example 2 using 1-methyl-piperazine.

Analytical HPLC: t_{R}=5.74 min (Grad 1); ES-MS: m/eₒ, =497.3.

### Example 7

### trans-5-(3-Benzyloxy-phenyl)-7-[4-(4-methyl-piperazin-1-yl)-cyclohexyl]-7H-pyrrolo[2,3-d]pyrimidin-4-ylamine

The title compound is obtained as described in Example 2 using cis-4-[4-amino-5-(3-benzyloxy-phenyl)-pyrrolo[2,3-d]pyrimidin-7-yl]-cyclohexanol (Example 1A) as starting material and 1-methyl-piperazine.

Analytical HPLC: t_{R}=5.78 min (Grad 1); ES-MS: m/eₒ, =497.2.

### Example 8

### cis-5-(3-Benzyloxy-phenyl)-7-(4-morpholinyl-cyclohexyl)-7H-pyrrolo[2,3-d]pyrimidin-4-ylamine

The title compound is obtained as described in Example 2 using morpholine.

Analytical HPLC: t_{R}=6.09 min (Grad 1); ES-MS: m/eₒ, =484.2.

### Example 9

### trans-5-(3-Benzyloxy-phenyl)-7-(4-morpholin-4-yl-cyclohexyl)-7H-pyrrolo[2,3-d]pyrimidin-4-ylamine

The title compound is obtained as described in Example 2 using cis-4-[4-amino-5-(3-benzyloxy-phenyl)-pyrrolo[2,3-d]pyrimidin-7-yl]-cyclohexanol (Example 1A) as starting material and morpholine.

Analytical HPLC: t_{R}=6.10 min (Grad 1); ES-MS: m/eₒ=484.2.

### Example 10

### cis-7-(4-Azetidin-1-yl-cyclohexyl)-5-(3-benzyloxy-phenyl)-7H-pyrrolo[2,3-d]pyrimidin-4-ylamine

The title compound is obtained as described in Example 2 using azetidine.

Analytical HPLC: t_{R}=6.54 min (Grad 1); ES-MS: m/eₒ=454.3.

### Example 11

### trans-7-(4-Azetidin-1-yl-cyclohexyl)-5-(3-benzyloxy-phenyl)-7H-pyrrolo[2,3-d]pyrimidin-4-ylamine

The title compound is obtained as described in Example 2 using cis-4-[4-amino-5-(3-benzyloxy-phenyl)-pyrrolo[2,3-d]pyrimidin-7-yl]-cyclohexanol (Example 1A) as starting material and azetidine.

Analytical HPLC: t_{R}=6.25 min (Grad 1); ES-MS: m/eₒ=454.2.

### Example 12

### cis-5-(3-Benzyloxy-phenyl)-7-(4-thiomorpholin-4-yl-cyclohexyl)-7H-pyrrolo[2,3-d]pyrimidin-4-ylamine

The title compound is obtained as described in Example 2 using thiomorpholine.

Analytical HPLC: t_{R}=6.31 min (Grad 1); ES-MS: m/eₒ=500.2.

### Example 13

### trans-5-(3-Benzyloxy-phenyl)-7-(4-thiomorpholin-4-yl-cyclohexyl)-7H-pyrrolo[2,3-d]pyrimidin-4-ylamine

The title compound is obtained as described in Example 2 using cis-4-[4-amino-5-(3-benzyloxy-phenyl)-pyrrolo[2,3-d]pyrimidin-7-yl]-cyclohexanol (Example 1A) as starting material and thiomorpholine.

Analytical HPLC: t_{R}=6.30 min (Grad 1); ES-MS: m/eₒ=500.1.

### Example 14

### trans-5-(3-Benzyloxy-phenyl)-7-(4-diethylamino-cyclohexyl)-7H-pyrrolo[2,3-d]pyrimidin-4-ylamine

The title compound is obtained as described in Example 2 using cis-4-[4-amino-5-(3-benzyloxy-phenyl)-pyrrolo[2,3-d]pyrimidin-7-yl]-cyclohexanol (Example 1A) as starting material and diethyl-amine.

Analytical HPLC: t_{R}=6.26 min (Grad 1); ES-MS: m/eₒ=470.3.

### Example 15A

### cis-7-(4-Amino-cyclohexyl)-5-(3-benzyloxy-phenyl)-7H-pyrrolo[2,3-d]pyrimidin-4-ylamine; and

### Example 15B

### trans-7-(4-Amino-cyclohexyl)-5-(3-benzyloxy-phenyl)-7H-pyrrolo[2,3-d]pyrimidin-4-ylamine

### Step 15.1: (4-Hydroxy-cyclohexyl)-carbamic acid tert-butyl ester

10 ml of cis/trans-4-amino-cyclohexanol (50% solution in water; Fluka, Buchs, Switzerland) and 11 ml of di-tert-butyl-dicarbonate (Fluka, Buchs, Switzerland) are added to 20 ml of 0.1 N NaOH. After stirring for 2 h at RT, the solution is extracted with petroleum ether and the organic phase is discarded. The aqueous phase is treated with 0.1 N HCl to pH=4 and extracted with ethyl acetate. The organic phase is dried over sodium sulfate and concentrated in vacuo to yield the title compound. R_{f} 0.47 (dichloromethane/methanol/water/acetic acid, 850:130:15:5, v/v/v/v).

### Step 15.2: Methanesulfonic acid 4-tert-butoxycarbonylamino-cyclohexyl ester

4.22 g of (4-hydroxy-cyclohexyl)-carbamic acid tert-butyl ester are dissolved in 25 ml of dichloromethane and 1.75 ml of methanesulfonyl chloride and 4.10 ml (29.4 mmol) of triethylamine are added. The solution is stirred for 30 min at 0° C. and 3 h at RT. Working-up is effected by partitioning between water and dichloromethane. The organic layer is concentrated in vacuo to yield the title compound that is used without further purification. R_{f} 0.63 (chloroform/methanol/water/acetic acid, 850:130:15:5, v/v/v/v).

### Step 15.3: cis/trans-{4-[4-Amino-5-(3-benzyloxy-phenyl)-pyrrolo[2,3-d]pyrimidin-7-yl]-cyclohexyl}carbamic acid tert-butyl ester

The title compound is obtained as described in Example 1, Step 1.2 using methanesulfonic acid 4-tert-butoxycarbonylamino-cyclohexyl ester. The crude compound is purified by flash chromatograpy (dichloromethane/methanol, 95:5, v/v) to yield the title compound. ES-MS: m/eₒ=514.0.

### Step 15.4: cis- and trans-7-(4-Amino-cyclohexyl)-5-(3-benzyloxy-phenyl)-7H-pyrrolo[2,3-d]pyrimidin-4-ylamine

3.3 g of cis/trans-{4-[4-amino-5-(3-benzyloxy-phenyl)-pyrrolo[2,3-d]pyrimidin-7-yl]-cyclohexyl}-carbamic acid tert-butyl ester are dissolved in 10 ml of formic acid and the solution is stirred for 1 h at 50° C. 100 ml of n-butanol are added and the organic phase is washed with 5% sodium bicarbonate and water. The organic phase is evaporated in vacuo and the crude product is purified by flash chromatography (dichloromethane/methanol, 4:2, v/v) to yield the two structural isomers of the title compound.
cis-7-(4-Amino-cyclohexyl)-5-(3-benzyloxy-phenyl)-7H-pyrrolo[2,3-d]pyrimidin-4-ylamine

Analytical HPLC: t_{R}=9.25 min (Grad 2); ES-MS: m/eₒ=414.1.
trans-7-(4-Amino-cyclohexyl)-5-(3-benzyloxy-phenyl)-7H-pyrrolo[2,3-d]pyrimidin-4-ylamine

Analytical HPLC: t_{R}=9.43 min (Grad 2); ES-MS: m/eₒ=414.1.

### Example 16

### cis-{4-[4-Amino-5-(3-benzyloxy-phenyl)-pyrrolo[2,3-d]pyrimidin-7-yl]-cyclohexyl}-carbamic acid methyl ester

165 mg of cis-7-(4-amino-cyclohexyl)-5-(3-benzyloxy-phenyl)-7H-pyrrolo[2,3-d]pyrimidin-4-ylamine (Example 15A) are dissolved in 5 ml of dichloromethane, and 39 μl of methyl chloroformate (Fluka, Buchs, Switzerland) and 10 μl of triethylamine are added to the solution. After stirring for 2 h at RT, the solution is evaporated in vacuo and the crude compound is purified by medium-pressure liquid chromatography.

Analytical HPLC: t_{R}=7.13 min (Grad 1); ES-MS: m/eₒ=472.0.

### Example 17

### cis-{4-[4-Amino-5-(3-benzyloxy-phenyl)-pyrrolo[2,3-d]pyrimidin-7-yl]-cyclohexyl}-3-methyl-urea

83 mg of cis-7-(4-amino-cyclohexyl)-5-(3-benzyloxy-phenyl)-7H-pyrrolo[2,3-d]pyrimidin-4-ylamine (Example 15A) and 14 mg of methylisocyanate (ChemService Inc., West Chester, Pa., U.S.A.) are added to 5 ml of acetonitrile. After stirring for 1 h at RT, the solution is concentrated in vacuo and the crude compound is purified by medium-pressure liquid chromatography.

Analytical HPLC: t_{R}=6.61 min (Grad 1); ES-MS: m/e₀=471.2.

### Example 18

### cis-N-{4-[4-Amino-5-(3-benzyloxy-phenyl)-pyrrolo[2,3-d]pyrimidin-7-yl]-cyclohexyl}-2-piperidin-1-yl-acetamide

### Step 18.1: cis-N-{4-[4-Amino-5-(3-benzyloxy-phenyl)-pyrrolo[2,3-d]pyrimidin-7-yl]-cyclohexyl}-2-chloro-acetamide

496 mg of cis-7-(4-amino-cyclohexyl)-5-(3-benzyloxy-phenyl)-7H-pyrrolo[2,3-d]pyrimidin-4-ylamine (Example 15A), 176 μl of triethylamine and 100 μl of chloroacetyl chloride (Fluka, Buchs, Switzerland) are added to 10 ml of acetonitrile. After stirring for 1 h at RT, the solution is concentrated in vacuo and the crude compound is purified by medium-pressure liquid chromatography.

Analytical HPLC: t_{R}=7.01 min (Grad 1); ES-MS: m/eₒ=489.9.

### Step 18.2: cis-N-{4-[4-Amino-5-(3-benzyloxy-phenyl)-pyrrolo[2,3-d]pyrimidin-7-yl]-cyclohexyl}-2-piperidin-1-yl-acetamide

105 mg of cis-N-{4-[4-amino-5-(3-benzyloxy-phenyl)-pyrrolo[2,3-d]pyrimidin-7-yl]-cyclohexyl}-2-chloro-acetamide and 85 μl of piperidine are added to 5 ml of ethanol. The solution is refluxed for 3 h. The solution is concentrated in vacuo and the crude compound is purified by medium-pressure liquid chromatography.

Analytical HPLC: t_{R}=6.15 min (Grad 1); ES-MS: m/e₀=538.9.

### Example 19

### cis-N-{4-[4-Amino-5-(3-benzyloxy-phenyl)-pyrrolo[2,3-d]pyrimidin-7-yl]-cyclohexyl}-2-morpholin-4-yl-acetamide

The title compound is obtained as described in Example 18 using morpholine.

Analytical HPLC: t_{R}=5.98 min (Grad 1); ES-MS: m/eₒ=540.9.

### Example 20

### cis-N-{4-[4-Amino-5-(3-benzyloxy-phenyl)-pyrrolo[2,3-d]pyrimidin-7-yl]-cyclohexyl}-2-(4-methyl-piperazin-1-yl)-acetamide

The title compound is obtained as described in Example 18 using 1-metyhl-piperazine.

Analytical HPLC: t_{R}=5.77 min (Grad 1); ES-MS: m/eₒ=554.0.

### Example 21

### cis-5-(3-Benzyloxy-phenyl)-7-[4-(pyrimidin-2-ylamino)-cyclohexyl]-7H-pyrrolo[2,3-d]pyrimidin-4-ylamine

413 mg of cis-7-(4-amino-cyclohexyl)-5-(3-benzyloxy-phenyl)-7H-pyrrolo[2,3-d]pyrimidin-4-ylamine (Example 15A), 175 mg of 2-bromopyrimidine (Fluka, Buchs, Switzerland) and 188 μl of diethylamine are added to 5 ml of dimethylformamide. The solution is stirred at 80° C. for 72 h. Working-up is effected by partitioning between water and ethyl acetate. The crude product is purified by medium-pressure liquid chromatography and flash chromatography (dichloromethane/methanol, 24/1, v/v).

Analytical HPLC: t_{R}=6.73 min (Grad 1); ES-MS: m/eₒ=492.2.

### Example 22

### cis-5-(3-Benzyloxy-phenyl)-7-[4-(1,4,5,6-tetrahydro-pyrimidin-2-ylamino)-cyclohexyl]-7H-pyrrolo[2,3-d]pyrimidin-4-ylamine

50 mg of cis-5-(3-benzyloxy-phenyl)-7-[4-(pyrimidin-2-ylamino)-cyclohexyl]-7H-pyrrolo[2,3-d]pyrimidin-4-ylamine (Example 21) are dissolved in 3 ml of ethyl acetate in the presence of 5 mg of PtO₂. After hydrogenation, the catalyst is removed by filtration and the solution is concentrated in vacuo. The crude product is purified by medium-pressure liquid chromatography.

Analytical HPLC: t_{R}=6.35 min (Grad 1); ES-MS: m/eₒ=496.3.

### Example 23

### cis-5-(3-Benzyloxy-phenyl)-7-[4-(4,5-dihydro-1H-imidazol-2-ylamino)-cyclohexyl]-7H-pyrrolo[2,3-d]pyrimidin-4-ylamine

207 mg of cis-7-(4-amino-cyclohexyl)-5-(3-benzyloxy-phenyl)-7H-pyrrolo[2,3-d]pyrimidin-4-ylamine (Example 15A) and 123 mg of 2-(methylthio)-2-imidazoline (Waco Chemicals, Ness, Germany) are dissolved in 4 ml of pyridine. After stirring for 16 h at 80° C., the solution is concentrated in vacuo. The crude product is purified by medium-pressure liquid chromatography.

Analytical HPLC: t_{R}=6.23 min (Grad 1); ES-MS: m/e₀=482.2.

### Example 24

### cis-N-{4-[4-Amino-5-(3-benzyloxy-phenyl)-pyrrolo[2,3-d]pyrimidin-7-yl]-cyclohexyl}methanesulfonamide

103 mg of cis-7-(4-amino-cyclohexyl)-5-(3-benzyloxy-phenyl)-7H-pyrrolo[2,3-d]pyrimidin-4-ylamine (Example 15A), 40.6 μl of methanesulfonyl chloride (Fluka, Buchs, Switzerland) and 70 μl of triethylamine are added to 5 ml of dichloromethane. The solution is stirred for 16 h at RT. Working-up is effected by partitioning between water and dichloromethane. The organic phase is dried over sodium sulfate and concentrated in vacuo to yield the title compound.

Analytical HPLC: t_{R}=6.90 min (Grad 1); ES-MS: m/eₒ=492.0.

### Example 25

### cis-N-{4-[4-Amino-5-(3-benzyloxy-phenyl)-pyrrolo[2,3-d]pyrimidin-7-yl]-cyclohexy}N,N-dimethylaminosulfonamide

The title compound is obtained as described in Example 24 using dimethylsulfamoylchloride (Fluka, Buchs, Switzerland) and acetonitrile as co-solvent.

Analytical HPLC: t_{R}=7.26 min (Grad 1); ES-MS: m/eₒ=521.0.

### Example 26

### cis-5-(3-Benzyloxy-phenyl)-7-(4-dimethylamino-cyclohexyl)-7H-pyrrolo[2,3-d]pyrimidin-4-ylamine

100 mg of cis-7-(4-amino-cyclohexyl)-5-(3-benzyloxy-phenyl)-7H-pyrrolo[2,3-d]pyrimidin-4-ylamine (Example 15A), 51 μl of formaldehyde 36% and 38 μl of formic acid 88% are dissolved in 2 ml of tetrahydrofurane. The solution is stirred at 80° C. for 3 h and concentrated in vacuo. The crude compound is purified by medium-pressure liquid chromatography.

Analytical HPLC: t_{R}=5.97 min (Grad 1); ES-MS: m/eₒ=442.0.

### Example 27

### N-{4-[4-Amino-5-(3-benzyloxy-phenyl)-pyrrolo[2,3-d]pyrimidin-7-yl]-cyclohexyl}-acetamide

70 mg of cis-7-(4-amino-cyclohexyl)-5-(3-benzyloxy-phenyl)-7H-pyrrolo[2,3-d]pyrimidin-4-ylamine (Example 15A) and 28 μl of acetic anhydride are added to 2 ml of tetrahydrofurane. The solution is stirred for 1 h at RT and concentrated in vacuo. The crude compound is purified by medium-pressure liquid chromatography.

Analytical HPLC: t_{R}=6.70 min (Grad 1); ES-MS: m/eₒ=456.0.

### Example 28

### cis-1-{4-[4-Amino-5-(3-benzyloxy-phenyl)-pyrrolo[2,3-d]pyrimidin-7-yl]-cyclohexyl}-3-ethyl-urea

103 mg of cis-7-(4-amino-cyclohexyl)-5-(3-benzyloxy-phenyl)-7H-pyrrolo[2,3-d]pyrimidin-ylamine (Example 15A) and 20 μl of ethyl isocyanate are added to 5 ml of acetonitrile. The solution is stirred for 3 h at RT and concentrated in vacuo. The crude compound is purified by medium-pressure liquid chromatography.

Analytical HPLC: t_{R}=6.85 min (Grad 1); ES-MS: m/e₀=485.0.

### Example 29

### cis-1-{4-[4-Amino-5-(3-benzyloxy-phenyl)-pyrrolo[2,3-d]pyrimidin-7-yl]- cyclohexyl}-3-isopropyl-urea

The title compound is obtained as described in Example 28 using isopropyl isocyanate.

Analytical HPLC: t_{R}=7.09 min (Grad 1); ES-MS: m/eₒ=499.0.

### Example 30

### cis-1-{4-[4-Amino-5-(3-benzyloxy-phenyl)-pyrrolo[2,3-d]pyrimidin-7-yl]-cyclohexy}-3-propyl-urea

The title compound is obtained as described in Example 28 using n-propyl isocyanate.

Analytical HPLC: t_{R}=7.09 min (Grad 1); ES-MS: m/eₒ=499.0.

### Example 31

### cis-1-{4-[4-Amino-5-(3-benzyloxy-phenyl)-pyrrolo[2,3-d]pyrimidin-7-yl]-cyclohexyl}-3-butyl-urea

The title compound is obtained as described in Example 28 using n-butyl isocyanate.

Analytical HPLC: t_{R}=7.34 min (Grad 1); ES-MS: m/eₒ=513.0.

### Example 32

### cis-{4-[4-Amino-5-(3-benzyloxy-phenyl)-pyrrolo[2,3-d]pyrimidin-7-yl]-cyclohexyl}-3-(3-methyl-benzyl)-urea

The title compound is obtained as described in Example 28 using 3-methylbenzyl isocyanate.

Analytical HPLC: t_{R}=7.00 min (Grad 1); ES-MS: m/eₒ=560.9.

### Example 33

### cis-1-{4-[4-Amino-5-(3-benzyloxy-phenyl)-pyrrolo[2,3-d]pyrimidin-7-yl]-cyclohexyl}-3-benzyl-urea

The title compound is obtained as described in Example 28 using benzyl isocyanate.

Analytical HPLC: t_{R}=7.37 min (Grad 1); ES-MS: m/eₒ=546.9.

### Example 34

### cis-1-{4-[4-Amino-5-(3-benzyloxy-phenyl)-pyrrolo[2.3-d]pyrimidin-7-yl]-cyclohexyl}-3-(4-methoxy-benzyl)-urea

The title compound is obtained as described in Example 28 using 4-methoxybenzyl isocyanate.

Analytical HPLC: t_{R}=7.33 min (Grad 1); ES-MS: m/eₒ=576.9.

### Example 35

### cis-1-{4-[4-Amino-5-(3-benzyloxy-phenyl)-pyrrolo[2,3-d]pyrimidin-7-yl]-cyclohexyl}-3-tert-butyl-urea

The title compound is obtained as described in Example 28 using tert-butyl isocyanate.

Analytical HPLC: t_{R}=7.44 min (Grad 1); ES-MS: m/eₒ=513.0.

### Example 36

### cis-N-{4-[4-Amino-5-(3-benzyloxy-phenyl)-pyrrolo[2,3-d]pyrimidin-7-yl]-cyclohexyl}-guanidine

124 mg of cis-7-(4-amino-cyclohexyl)-5-(3-benzyloxy-phenyl)-7H-pyrrolo[2,3-d]pyrimidin-4-ylamine (Example 15A) and 85 mg of N,N′-bis-tert.-butoxycarbonyl-1-guanylpyrazole (Advanced ChemTech Europe, Machelen, Belgium) are dissolved in 5 ml of acetonitrile. After 2 h of stirring at RT, the solution is concentrated in vacuo. The residue is dissolved in 5 ml of formic acid and the solution is stirred for 1 h at 50° C. The crude product is purified by medium-pressure liquid chromatography to provide the title compound.

Analytical HPLC: t_{R}=6.12 min (Grad 1); ES-MS: m/eₒ=456.0.

### Example 37

### cis-1-{4-[4-Amino-5-(3-benzyloxy-phenyl)-pyrrolo[2,3-d]pyrimidin-7-yl]-cyclohexyl}-3-(2-dimethylamino-ethyl)-urea

### Step 37.1: cis-1-{4-[4-Amino-5-(3-benzyloxy-phenyl)-pyrrolo[2,3-d]pyrimidin-7-yl]-cyclohexyl}-3-(2-bromo-ethyl)-urea

The title compound is prepared as described in Example 28 using 2-bromoethyl isocyanate.

Analytical HPLC: t_{R}=7.12 min (Grad 1); ES-MS: m/eₒ=562.8, 564.8, 565.8.

### Step 37.2: cis-1-{4-[4-Amino-5-(3-benzyloxy-phenyl)-pyrrolo[2,3-d]pyrimidin-7-yl]-cyclohexyl}-3-(2-dimethylamino-ethyl)-urea

48 mg of cis-1-{4-[4-amino-5-(3-benzyloxy-phenyl)-pyrrolo[2,3-d]pyrimidin-7-yl]-cyclohexyl}-3-(2-bromo-ethyl)-urea and 90 μl of 5.6 M dimethylamine in ethanol are dissolved in 4 ml of ethanol. The solution is stirred for five days. The crude compound is purified by medium-pressure liquid chromatography to provide the title compound.

Analytical HPLC: t_{R}=6.03 min (Grad 1); ES-MS: m/eₒ=527.9.

### Example 38

### cis-1-{4-[4-Amino-5-(3-benzyloxy-phenyl)-pyrolo[2,3-d]pyrimidin-7-yl]-cyclohexyl}-3-(2-morpholin-4-yl-ethyl)-urea

The title compound is obtained as described in Example 37 using morpholine.

Analytical HPLC: t_{R}=6.07 min (Grad 1); ES-MS: m/eₒ=569.9.

### Example 39

### cis-1-{4-[4-Amino-5-(3-benzyloxy-phenyl)-pyrrolo[2,3-d]pyrimidin-7-yl]cyclohexyl}-3-(3-morpholin-4-yl-propyl)-urea

### Step 39.1: cis-1-{4-[4-Amino-5-(3-benzyloxy-phenyl)-pyrrolo[2,3-d]pyrimidin-7-yl]-cyclohexyl}-3-(3-chloro-propyl)-urea

The title compound is prepared as described in Example 28 using 3-chloropropyl isocyanate.

Analytical HPLC: t_{R}=7.19 min (Grad 1); ES-MS: m/eₒ=532.9.

### Step 39.2: cis-1-{4-[4-Amino-5-(3-benzyloxy-phenyl)-pyrrolo[2,3-d]pyrimidin-7-yl]-cyclohexyl}-3-(3-morpholin-4-yl-propyl)-urea

The title compound is prepared as described in Example 37 using cis-1-{4-[4-amino-5-(3-benzyloxy-phenyl)-pyrrolo[2,3-d]pyrimidin-7-yl]-cyclohexyl}-3-(3-chloro-propyl)-urea and morpholine.

Analytical HPLC: t_{R}=6.07 min (Grad 1); ES-MS: m/eₒ=583.9.

### Example 40

### cis-{4-[4-Amino-5-(3-benzyloxy-phenyl)-pyrrolo[2,3-d]pyrimidin-7-yl]-gyclohexyl}-carbamic acid 2-methoxy-ethyl ester

103 mg of cis-7-(4-amino-cyclohexyl)-5-(3-benzyloxy-phenyl)-7H-pyrrolo[2,3-d]pyrimidin-4-ylamine (Example 15A), 42 mg of 2-methxoyethyl chloroformate and 42 ml of triethylamine are dissolved in 5 ml of dichloromethane. The solution is stirred for 2 h at RT. The crude product is purified by medium-pressure liquid chromatography to provide the title compound.

Analytical HPLC: t_{R}=7.13 min (Grad 1); ES-MS: m/eₒ=516.0.

### Example 41

### cis-4-[4-Amino-5-(3-benzyloxy-phenyl)-6-bromo-pyrrolo[2,3-d]pyrimidin-7-yl]-cyclohexanol

1.02 of cis-4-[4-amino-5-(3-benzyloxy-phenyl)-pyrrolo[2,3-d]pyrimidin-7-yl]-cyclohexanol (Example 1A) and 0.50 g of N-bromosuccinimide are dissolved in 20 ml of dimethylformamide. The solution is stirred for 30 min at RT and working-up is effected by partitioning between water and ethyl acetate. The organic phase is concentrated in vacuo and the crude compound is purified by by flash column chromatography (dichloromethane/methanol, 95:5, v/v).

Analytical HPLC: t_{R}=7.51 min (Grad 1); ES-MS: m/eₒ=495.0.

### Example 42

### trans-4-[4-Amino-5-(3-benzyloxy-phenyl)-6-bromo-pyrrolo[2,3-d]pyrimidin-7-yl]-cyclohexanol

The title compound is obtained as described in Example 41 using trans-4-[4-amino-5-(3-benzyloxy-phenyl)-pyrrolo[2,3-d]pyrimidin-7-yl]-cyclohexanol (Example 1B) as starting material.

Analytical HPLC: t_{R}=7.26 min (Grad 1); ES-MS: m/eₒ=495.1.

### Example 43

### cis-4-[4-Amino-5-(3-benzyloxy-phenyl)-6-methyl-pyrrolo[2,3-d]pyrimidin-7-yl]-cyclohexanol

In a sealed tube, 540 mg of cis-4-[4-amino-5-(3-benzyloxy-phenyl)-6-bromo-pyrrolo[2,3-d]pyrimidin-7-yl]-cyclohexanol (Example 41), 252 mg of tetrakis(triphenylphosphine) palladium (O) and 0.76 ml of tin tetramethyl (Fluka, Buchs, Switzerland) are heated under argon in 10 ml dry dimethylformamide for 2 h at 100-105° C. bath temperature. The reaction mixture is filtered and the residue is washed with dimethylformamide. Working-up is effected by partitioning between water and ethyl acetate. The organic phase is concentrated in vacuo and the crude compound is purified by medium-pressure liquid chromatography.

Analytical HPLC: t_{R}=7.38 min (Grad 1); ES-MS: m/eₒ=429.2.

### Example 44

### trans-4-[4-Amino-5-(3-benzyloxy-phenyl)-6-methyl-pyrrolo[2,3-d]pyrimidin-7-yl]-cyclohexanol

The title compound is obtained as described in Example 43 using trans-4-[4-amino-5-(3-benzyloxy-phenyl)-6-bromo-pyrrolo[2,3-d]pyrimidin-7-yl]-cyclohexanol (Example 42) as starting material.

Analytical HPLC: t_{R}=7.15 min (Grad 1); ES-MS: m/eₒ=429.3.

### Example 45

### trans-5-(3-Benzyloxy-phenyl)-6-methyl-7-[4-(4-methyl-piperazin-1-yl)-cyclohexyl]-7H-pyrrolo[2,3-d]pyrimidin-4-ylamine

The title compound is obtained as described in Example 2 using cis-4-[4-amino-5-(3-benzyloxy-phenyl)-6-methyl-pyrrolo[2,3-d]pyrimidin-7-yl]-cyclohexanol (Example 43) and 1-methylpiperazine as staring materials.

Analytical HPLC: t_{R}=6.44 min (Grad 1); ES-MS: m/eₒ=511.3.

### Example 46

### trans-5-(3-Benzyloxy-phenyl)-7-(4-dimethylamino-cyclohexyl)-6-methyl-7H-pyrrolo[2,3-d]pyrimidin-4-ylamine

The title compound is obtained as described in Example 2 using cis-4-[4-amino-5-(3-benzyloxy-phenyl)-6-methyl-pyrrolo[2,3-d]pyrimidin-7-yl]-cyclohexanol (Example 43) and dimethylamine.

Analytical HPLC: t_{R}=6.77 min (Grad 1); ES-MS: m/eₒ=456.3.

### Example 47

### trans-5-(3-Benzyloxy-phenyl)-7-(4-diethylamino-cyclohexyl)-6-methyl-7H-Pyrrolo[2,3-d]pyrimidin-4-ylamine

The title compound is obtained as described in Example 2 using cis-4-[4-amino-5-(3-benzyloxy-phenyl)-6-methyl-pyrrolo[2,3-d]pyrimidin-7-yl]-cyclohexanol (Example 43) and diethylamine.

Analytical HPLC: t_{R}=6.89 min (Grad 1); ES-MS: m/eₒ=484.3.

### Example 48

### trans-5-(3-Benzyloxy-phenyl)-6-methyl-7-(4-pyrrolidin-1-yl-cyclohexyl)-7H-Pyrrolo[2,3-d]pyrimidin-4-ylamine

The title compound is obtained as described in Example 2 using cis-4-[4-amino-5-(3-benzyloxy-phenyl)-6-methyl-pyrrolo[2,3-d]pyrimidin-7-yl]-cyclohexanol (Example 43) and pyrrolidine.

Analytical HPLC: t_{R}=6.84 min (Grad 1); ES-MS: m/eₒ=482.3.

### Example 49

### trans-5-(3-Benzyloxy-phenyl)-6-methyl-7-(4-morpholin-4-yl-cyclohexyl)-7H-Pyrrolo[2,3-d]pyrimidin-4-ylamine

The title compound is obtained as described in Example 2 using cis-4-[4-amino-5-(3-benzyloxy-phenyl)-6-methyl-pyrrolo[2,3-d]pyrimidin-7-yl]-cyclohexanol (Example 43) and morpholine.

Analytical HPLC: t_{R}=6.60 min (Grad 1); ES-MS: m/eₒ=498.2.

### Example 50

### trans-7-(4-Azetidin-1-yl-cyclohexyl)-5-(3-benzyloxy-phenyl)-6-methyl-7H-Pyrrolo[2,3-d]pyrimidin-4-ylamine

The title compound is obtained as described in Example 2 using cis-4-[4-amino-5-(3-benzyloxy-phenyl)-6-methyl-pyrrolo[2,3-d]pyrimidin-7-yl]-cyclohexanol (Example 43) and azetidine. Analytical HPLC: t_{R}=6.67 min (Grad 1); ES-MS: m/eₒ=468.2.

### Example 51

### Test for activity against IGF-I induced IGF-IR autophosphorylation using the cellular “Capture ELISA” test

The cellular “Capture ELISA” test is carried out as described above. The IC₅₀ values for some of the compounds of the present invention are given below:

| Compound of Example | IC₅₀ (μM) |
|---|---|
| 4 | 0.39 |
| 5 | 0.46 |
| 10 | 0.12 |
| 11 | 0.35 |
| 47 | 0.40 |
| 48 | 0.11 |

### Example 52

### Tablets

Tablets comprising 50 mg of active ingredient, for example one of the compounds of formula I described in Examples 1 to 50, and having the following composition are prepared in customary manner:

Composition

| | | |
|---|---|---|
| active ingredient | 50 | mg |
| wheat starch | 150 | mg |
| lactose | 125 | mg |
| colloidal silicic acid | 12.5 | mg |
| talc | 22.5 | mg |
| magnesium stearate | 2.5 | mg |
| Total: | 362.5 | mg |

Preparation: The active ingredient is mixed with a portion of the wheat starch, with the lactose and the colloidal silicic acid and the mixture is forced through a sieve. A further portion of the wheat starch is made into a paste, on a water bath, with five times the amount of water and the powder mixture is kneaded with the paste until a slightly plastic mass is obtained.

The plastic mass is pressed through a sieve of about 3 mm mesh size and dried, and the resulting dry granules are again forced through a sieve. Then the remainder of the wheat starch, the talc and the magnesium stearate are mixed in and the mixture is compressed to form tablets weighing 145 mg and having a breaking notch.

### Example 53

### Soft Capsules

5000 soft gelatin capsules comprising each 50 mg of active ingredient, for example one of the compounds of formula I described in Examples 1 to 50, are prepared in customary manner:

Composition:

| | | |
|---|---|---|
| active ingredient | 250 | g |
| Lauroglykol | 2 | litres |

Preparation: The pulverized active ingredient is suspended in Lauroglykol® (propylene glycol laurate, Gattefossé S. A., Saint Priest, France) and ground in a wet pulverizer to a particle size of approx. 1 to 3 μm. 0.419 g portions of the mixture are then dispensed into soft gelatin capsules using a capsule-filling machine.

1. A compound of formula I 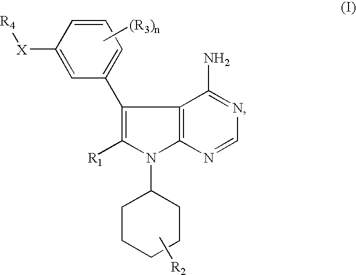
wherein
n is from 0 to 4,
R₁ is hydrogen, unsubstituted or substituted lower alkyl or halogen,
R₂ is hydroxy; unsubstituted, mono- or disubstituted amino; a heterocyclic radical containing at least one nitrogen ring atom and being-attached to the cyclohexane ring of the molecule of formula I via a nitrogen ring atom; a radical R₅—(C═Y)—NH—, wherein R₅ is unsubstituted or substituted lower alkyl, unsubstituted, mono- or disubstituted amino, a heterocyclic radical, or etherified hydroxy, and Y is oxygen, sulfur or imino; or a radical R₆-sulfonylamino, wherein
R₆ is unsubstituted or substituted lower alkyl, unsubstituted, mono- or disubstituted amino or phenyl optionally substituted by lower alkyl, lower alkoxy or nitro,
R₃ is lower alkyl, hydroxy-, amino- or halogen-substituted lower alkyl, hydroxy, cyano, lower alkoxy, lower alkanoyl, lower alkanoyloxy, amino, mono- or di-lower alkylamino, lower alkanoylamino, carboxy, lower alkoxycarbonyl or halogen, wherein the R₃ substituents can be selected independently of one another if n>1,
R₄ is a radical R₇—CR₈(R₉)—, wherein R₇ is cyclobutyl, cyclopentyl, cyclohexyl, phenyl, furyl, pyrrolyl, thienyl or pyridyl, said R₇ substitutents being optionally substituted by one or more radicals selected from lower alkyl and halogen, and R₈ and R₉ are independently of each other hydrogen, lower alkyl or halogen, and
X is selected from —O—, —NH— and —S—,
or a salt thereof.

2. A compound of formula I according to claim 1, wherein
n is from 0 to 4,
R₁ is hydrogen, unsubstituted or substituted lower alkyl or halogen,
R₂ is hydroxy; unsubstituted, mono- or disubstituted amino; a heterocyclic radical containing at least one nitrogen ring atom and being attached to the cyclohexane ring of the molecule of formula I via a nitrogen ring atom; a radical R₅—(C═Y)—NH—, wherein R₅ is unsubstituted or substituted lower alkyl, unsubstituted, mono- or disubstituted amino, a heterocyclic radical, or etherified hydroxy, and Y is oxygen, sulfur or imino; or a radical R₆-sulfonylamino, wherein
R₆ is unsubstituted or substituted lower alkyl, unsubstituted, mono- or disubstituted amino or phenyl optionally substituted by lower alkyl, lower alkoxy or nitro,
R₃ is lower alkyl or lower alkoxy, wherein the R₃ substituents can be selected independently of one another if n>1,
R₄ is a radical R₇—CR₈(R₉)—, wherein R₇ is cyclobutyl, cyclopentyl, cyclohexyl, phenyl, furyl, pyrrolyl, thienyl, pyridyl or phenyl substituted by one or more radicals selected from lower alkyl and halogen, and R₈ and R₉ are independently of each other hydrogen, lower alkyl or halogen, and
X is selected from —O—, —NH— and —S—,
or a salt thereof.

3. A compound of formula I according to claim 1, wherein
n is 0,
R₁ is hydrogen, unsubstituted or substituted lower alkyl or halogen,
R₂ is hydroxy; unsubstituted, mono- or disubstituted amino; a heterocyclic radical containing at least one nitrogen ring atom and being attached to the cyclohexane ring of the molecule of formula I via a nitrogen ring atom; a radical R₅—(C═Y)—NH—, wherein R₅ is unsubstituted or substituted lower alkyl, unsubstituted, mono- or disubstituted amino, a heterocyclic radical, or etherified hydroxy, and Y is oxygen, sulfur or imino; or a radical R₆-sulfonylamino, wherein
R₆ is unsubstituted or substituted lower alkyl, unsubstituted, mono- or disubstituted amino or phenyl optionally substituted by lower alkyl, lower alkoxy or nitro,
R₄ is benzyl, and
X is selected from —O—, —NH— and —S—,
or a salt thereof.

4. A compound of formula I according to claim 1, wherein
n is 0,
R₁ is hydrogen, unsubstituted or substituted lower alkyl or halogen,
R₂ is hydroxy; unsubstituted, mono- or disubstituted amino; a heterocyclic radical having from 4 to 8 ring members and from 1 to 3 heteroatoms whereby at least one heteroatom is nitrogen and the binding of the heterocyclic radical to the cyclohexane ring of the molecule of formula I occurs via a nitrogen ring atom; a radical R₅—(C═Y)—NH—, wherein R₅ is lower alkyl, unsubstituted, mono- or disubstituted amino, etherified hydroxy, a heterocyclic radical having from 4 to 8 ring members and from 1 to 3 heteroatoms whereby at least one heteroatom is nitrogen and the binding of the heterocyclic radical occurs via a nitrogen ring atom, lower alkyl substituted by said heterocyclic radical or by one or more radicals selected independently of one another from the group consisting of amino, N-lower alkylamino, N,N-di-lower alkylamino, N-lower alkanoylamino, N,N-di-lower alkanoylamino, hydroxy, lower alkoxy, lower alkoxy-lower alkoxy, lower alkanoyl, lower alkanoyloxy, cyano, nitro, carboxy, lower alkoxycarbonyl, carbamoyl, amidino, guanidino, ureido, mercapto, lower alkylthio and halogen, and Y is oxygen, sulfur or imino; or a radical R₆-sulfonylamino, wherein R₆ is unsubstituted or substituted lower alkyl, unsubstituted, mono- or disubstituted amino or phenyl optionally substituted by lower alkyl, lower alkoxy or nitro,
R₄ is benzyl, and
X is selected from —O—, —NH— and —S—,
or a salt thereof.

5. A compound of formula I according to claim 1, wherein
n is 0,
R₁ is hydrogen, lower alkyl or halogen,
R₂ is hydroxy; unsubstituted, mono- or disubstituted amino; a heterocyclic radical having from 4 to 8 ring members and from 1 to 3 heteroatoms whereby at least one heteroatom is nitrogen and the binding of the heterocyclic radical to the cyclohexane ring of the molecule of formula I occurs via a nitrogen ring atom; a radical R₅—(C═Y)—NH—, wherein R₅ is lower alkyl, unsubstituted or monosubstituted amino, etherified hydroxy, or lower alkyl substituted by a heterocyclic radical having from 4 to 8 ring members and from 1 to 3 heteroatoms whereby at least one heteroatom is nitrogen and the binding of the heterocyclic radical occurs via a nitrogen ring atom, and Y is oxygen or imino; or a radical R₆-sulfonylamino, wherein R₆ is lower alkyl or disubstituted amino,
R₄ is benzyl, and
X is selected from —O—, —NH— and —S—,
or a salt thereof.

6. A compound of formula I according to claim 1, wherein
n is 0,
R₁ is hydrogen, lower alkyl or halogen,
R₂ is hydroxy, amino, N,N-di-lower alkylamino, pyrimidinyl-amino, 1,4,5,6-tetrahydro-pyrimidinyl-amino, 4,5-dihydro-1H-imidazolyl-amino, azetidin-1-yl, pyrrolidin-1-yl, 1-piperidyl, lower alkyl-piperazin-1-yl, morpholin-4-yl, thiomorpholin-4-yl; a radical R₅—(C═Y)—NH—, wherein R₅ is lower alkyl, lower alkoxy, amino, N-lower alkylamino, N-(phenyl-lower alkyl)-amino, N-(lower alkyl-phenyl-lower alkyl)-amino, N-(lower alkoxy-phenyl-lower alkyl)-amino, N-(morpholin-4-yl-lower alkyl)-amino, N-(N′,N′-di-lower alkylamino-lower alkyl)-amino, lower alkoxy-lower alkoxy, 1-piperidyl-lower alkyl, morpholin-4-yl-lower alkyl or lower alkyl-piperazin-1-yl-lower alkyl, and Y is oxygen or imino; or a radical R₆-sulfonylamino, wherein
R₆ is lower alkyl or N,N-di-lower alkylamino,
R₄ is benzyl, and
X is —O—,
or a salt thereof.

7. A compound of formula I according to claim 1, selected from the group consisting of
cis-4-[4-amino-5-(3-benzyloxy-phenyl)-pyrrolo[2,3-d]pyrimidin-7-yl]-cyclohexanol;
trans-4-[4-amino-5-(3-benzyloxy-phenyl)-pyrrolo[2,3-d]pyrimidin-7-yl]-cyclohexanol;
cis-5-(3-benzyloxy-phenyl)-7-(4-piperidin-1-yl-cyclohexyl)-7H-pyrrolo[2,3-d]pyrimidin-4-ylamine;
trans-5-(3-benzyloxy-phenyl)-7-(4-piperidin-1-yl-cyclohexyl)-7H-pyrrolo[2,3-d]pyrimidin-4-ylamine;
cis-5-(3-benzyloxy-phenyl)-7-(4-pyrrolidin-1-yl-cyclohexyl)-7H-pyrrolo[2,3-d]pyrimidin-4-ylamine;
trans-5-(3-benzyloxy-phenyl)-7-(4-pyrrolidin-1-yl-cyclohexyl)-7H-pyrrolo[2,3-d]pyrimidin-4-ylamine;
cis-5-(3-benzyloxy-phenyl)-7-[4-(4-methyl-piperazin-1-yl)-cyclohexyl]-7H-pyrrolo[2,3-d]pyrimidin-4-ylamine;
trans-5-(3-benzyloxy-phenyl)-7-[4-(4-methyl-piperazin-1-yl)-cyclohexyl]-7H-pyrrolo[2,3-d]pyrimidin-4-ylamine;
cis-5-(3-benzyloxy-phenyl)-7-(4-morpholin-4-y-cyclohexyl)-7H-pyrrolo[2,3-d]pyrimidin-4-ylamine;
trans-5-(3-benzyloxy-phenyl)-7-(4-morpholin-4-yl-cyclohexyl)-7H-pyrrolo[2,3-d]pyrimidin-4-ylamine;
cis-7-(4-azetidin-1-yl-cyclohexyl)-5-(3-benzyloxy-phenyl)-7H-pyrrolo[2,3-d]pyrimidin-4-ylamine;
trans-7-(4-azetidin-1-yl-cyclohexyl)-5-(3-benzyloxy-phenyl)-7H-pyrrolo[2,3-d]pyrimidin-4-ylamine;
cis-5-(3-benzyloxy-phenyl)-7-(4-thiomorpholin-4-yl-cyclohexyl)-7H-pyrrolo[2,3-d]pyrimidin-4-ylamine;
trans-5-(3-benzyloxy-phenyl)-7-(4-thiomorpholin-4-yl-cyclohexyl)-7H-pyrrolo[2,3-d]pyrimidin-4-ylamine;
trans-5-(3-benzyloxy-phenyl)-7-(4-diethylamino-cyclohexyl)-7H-pyrrolo[2,3-d]pyrimidin-4-ylamine;
cis-7-(4-amino-cyclohexyl)-5-(3-benzyloxy-phenyl)-7H-pyrrolo[2,3-d]pyrimidin-4-ylamine;
trans-7-(4-amino-cyclohexyl)-5-(3-benzyloxy-phenyl)-7H-pyrrolo[2,3-d]pyrimidin-4-ylamine;
cis-{4-[4-amino-5-(3-benzyloxy-phenyl)-pyrrolo[2,3-d]pyrimidin-7-yl]-cyclohexyl}-carbamic acid methyl ester;
cis-1-{4-[4-amino-5-(3-benzyloxy-phenyl)-pyrrolo[2,3-d]pyrimidin-7-yl]-cyclohexyl}-3-methyl-urea;
cis-N-{4-[4-amino-5-(3-benzyloxy-phenyl)-pyrrolo[2,3-d]pyrimidin-7-yl]-cyclohexyl}-2-piperidin-1-yl-acetamide;
cis-N-{4-[4-amino-5-(3-benzyloxy-phenyl)-pyrrolo[2,3-d]pyrimidin-7-yl]-cyclohexyl)-2-morpholin-4-yl-acetamide;
cis-N-{4-[4-amino-5-(3-benzyloxy-phenyl)-pyrrolo[2,3-d]pyrimidin-7-yl]-cyclohexyl}-2-(4-methyl-piperazin-1-yl)-acetamide;
cis-5-(3-benzyloxy-phenyl)-7-[4-(pyrimidin-2-ylamino)-cyclohexyl]-7H-pyrrolo[2,3-d]pyrimidin-4-ylamine;
cis-5-(3-benzyloxy-phenyl)-7-[4-(1,4,5,6-tetrahydro-pyrimidin-2-ylamino)-cyclohexyl]-7H-pyrrolo[2,3-d]pyrimidin-4-ylamine;
cis-5-(3-benzyloxy-phenyl)-7-[4-(4,5-dihydro-1H-imidazol-2-ylamino)-cyclohexyl]-7H-pyrrolo[
2,3-d]pyrimidin-4-ylamine;
cis-N-{4-[4-amino-5-(3-benzyloxy-phenyl)-pyrrolo[2,3-d]pyrimidin-7-yl]-cyclohexyl}-methanesulfonamide;
cis-N-{4-[4-amino-5-(3-benzyloxy-phenyl)-pyrrolo[2,3-d]pyrimidin-7-yl]-cyclohexyl}-N,N-dimethylaminosulfonamide;
cis-5-(3-benzyloxy-phenyl)-7-(4-dimethylamino-cyclohexyl)-7H-pyrrolo[2,3-d]pyrimidin-4-ylamine;
N-4-[4-amino-5-(3-benzyloxy-phenyl)-pyrrolo[2,3-d]pyrimidin-7-yl]-cyclohexyl}-acetamide;
cis-1-{4-[4-amino-5-(3-benzyloxy-phenyl)-pyrrolo[2,3-d]pyrimidin-7-yl]-cyclohexyl}-3-ethyl-urea;
cis-1-{4-[4-amino-5-(3-benzyloxy-phenyl)-pyrrolo[2,3-d]pyrimidin-7-yl]-cyclohexyl}-3-isopropyl-urea;
cis-1-{4-[4-amino-5-(3-benzyloxy-phenyl)-pyrrolo[2,3-d]pyrimidin-7-yl]-cyclohexyl}-3-propyl-urea;
cis-1-{4-[4-amino-5-(3-benzyloxy-phenyl)-pyrrolo[2,3-d]pyrimidin-7-yl]-cyclohexyl}-3-butyl-urea;
cis-1-{4-[4-amino-5-(3-benzyloxy-phenyl)-pyrrolo[2,3-d]pyrimidin-7-yl]-cyclohexyl}-3-(3-methyl-benzyl)-urea;
cis-1-{4-[4-amino-5-(3-benzyloxy-phenyl)-pyrrolo[2,3-d]pyrimidin-7-yl]-cyclohexyl}-3-benzyl-urea;
cis-1-{4-[4-amino-5-(3-benzyloxy-phenyl)-pyrrolo[2,3-d]pyrimidin-7-yl]-cyclohexyl}-3-(4-methoxy-benzyl)-urea;
cis-1-{4-[4-amino-5-(3-benzyloxy-phenyl)-pyrrolo[2,3-d]pyrimidin-7-yl]-cyclohexyl}-3-tert-butyl-urea;
cis-N-{4-[4-amino-5-(3-benzyloxy-phenyl)-pyrrolo[2,3-d]pyrimidin-7-yl]-cyclohexyl}-guanidine;
cis-1-{4-[4-amino-5-(3-benzyloxy-phenyl)-pyrrolo[2,3-d]pyrimidin-7-yl]-cyclohexyl}-3-(2-dimethylamino-ethyl)-urea;
cis-1-{4-[4-amino-5-(3-benzyloxy-phenyl)-pyrrolo[2,3-d]pyrimidin-7-yl]-cyclohexyl}-3-(2-morpholin-4-yl-ethyl)-urea;
cis-1-{4-[4-amino-5-(3-benzyloxy-phenyl)-pyrrolo[2,3-d]pyrimidin-7-yl]-cyclohexyl}-3-(3-morpholin-4-yl-propyl)-urea;
cis-{4-[4-amino-5-(3-benzyloxy-phenyl)-pyrrolo[2,3-d]pyrimidin-7-yl]-cyclohexyl}-carbamic acid 2-methoxy-ethyl ester;
cis-4-[4-amino-5-(3-benzyloxy-phenyl)-6-bromo-pyrrolo[2,3-d]pyrimidin-7-yl]-cyclohexanol;
trans-4-[4-amino-5-(3-benzyloxy-phenyl)-6-bromo-pyrrolo[2,3-d]pyrimidin-7-yl]-cyclohexanol;
cis-4-[4-amino-5-(3-benzyloxy-phenyl)-6-methyl-pyrrolo[2,3-d]pyrimidin-7-yl]-cyclohexanol;
trans-4-[4-amino-5-(3-benzyloxy-phenyl)-6-methyl-pyrrolo[2,3-d]pyrimidin-7-yl]-cyclohexanol;
trans-5-(3-benzyloxy-phenyl)-6-methyl-7-[4-(4-methyl-piperazin-1-yl)-cyclohexyl]-7H-pyrrolo[2,3-d]pyrimidin-4-ylamine;
trans-5-(3-benzyloxy-phenyl)-7-(4-dimethylamino-cyclohexyl)-6-methyl-7H-pyrrolo[2,3-d]pyrimidin-4-ylamine;
trans-5-(3-benzyloxy-phenyl)-7-(4-diethylamino-cyclohexyl)-6-methyl-7H-pyrrolo[2,3-d]pyrimidin-4-ylamine;
trans-5-(3-benzyloxy-phenyl)-6-methyl-7-(4-pyrrolidin-1-yl-cyclohexyl)-7H-pyrrolo[2,3-d]pyrimidin-4-ylamine;
trans-5-(3-benzyloxy-phenyl)-6-methyl-7-(4-morpholin-4-yl-cyclohexyl)-7H-pyrrolo[2,3-d]pyrimidin-4-ylamine;
trans-7-(4-azetidin-1-yl-cyclohexyl)-5-(3-benzyloxy-phenyl)-6-methyl-7H-pyrrolo[2,3-d]pyrimidin-4-ylamine;
and pharmaceutically acceptable salts thereof.

8. A compound of formula I, or a pharmaceutically acceptable salt thereof, according to claim 1 for use in a method for the treatment of the human or animal body.

9. A pharmaceutical composition comprising a compound of formula I or a pharmaceutically acceptable salt thereof according to claim 1, together with at least one pharmaceutically acceptable carrier.

10. A method for the treatment of a disease which responds to an inhibition of IGF-IR-dependent cell proliferation comprising administering a compound of formula I according to claim 1, or a pharmaceutically acceptable salt thereof.

11. A method for the treatment of a disease which responds to an inhibition of IGF-IR tyrosine kinase comprising administering a compound of formula I according to claim 1, or a pharmaceutically acceptable salt thereof.

12. A process for the preparation of a compound of formula I according to claim 1 or of a salt of such a compound, characterized in that
a) in order to prepare a compound of formula I, in which R₂ is hydroxy, a compound of formula II 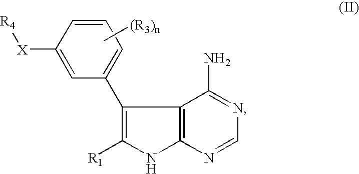
wherein n, R₁, R₃, R₄ and X have the meanings as defined for a compound of formula I, is reacted with methanesulfonic acid hydroxy-cyclohexyl ester;
b) in order to prepare a compound of formula I, in which R₂ is amino, a compound of formula II, wherein n, R₁, R₃, R₄ and X have the meanings as defined for a compound of formula I, is reacted in a first step with a compound of formula III 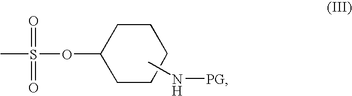
wherein PG is an amino protecting group which is removed in a second step;
c) in order to prepare a compound of formula I, in which R₂ is mono- or disubstituted amino or a heterocyclic radical containing at least one nitrogen ring atom and being attached to the cyclohexane ring of the molecule of formula I via a nitrogen ring atom, a compound of formula IV, 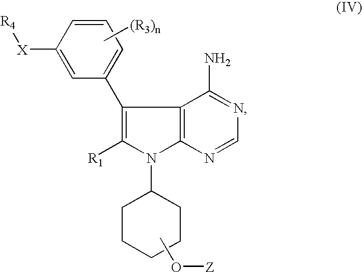
wherein n, R₁, R₃, R₄ and X have the meanings as defined for a compound of formula I and —O—Z is a leaving group, is reacted with a compound of the formula R₁₀—H in which R₁₀ is mono- or disubstituted amino or a heterocyclic radical containing at least one nitrogen ring atom wherein the heterocyclic radical is attached to the hydrogen atom of R₁₀—H via a nitrogen ring atom;
d) in order to prepare a compound of formula I, in which R₂ is a radical R₅—(C═Y)—NH— wherein R₅ is unsubstituted or substituted lower alkyl and Y is oxygen, a compound of formula I, in which R₂ is amino, is reacted with a compound of the formula R₅—(C═O)-Halogen wherein R₅ is unsubstituted or substituted lower alkyl;
e) in order to prepare a compound of formula I, in which R₂ is a radical R₅—(C═Y)—NH— wherein R₅ is lower alkyl substituted by a heterocyclic radical containing at least one nitrogen ring atom whereby the binding of the heterocyclic radical to lower alkyl occurs via a nitrogen ring atom, and Y is oxygen, a compound of formula V 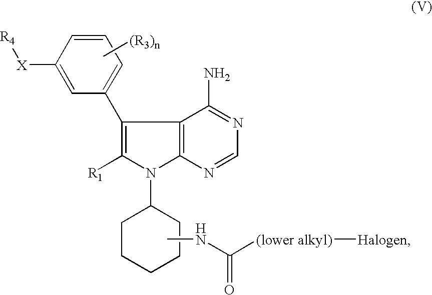
wherein n, R₁, R₃, R₄ and X have the meanings as defined for a compound of formula I, is reacted with a compound of the formula R₁₁—H in which R₁₁ is a heterocyclic radical containing at least one nitrogen ring atom wherein the heterocyclic radical is attached to the hydrogen atom of R₁, —H via a nitrogen ring atom;
f) in order to prepare a compound of formula I, in which R₂ is a radical R₅—(C═Y)—NH— wherein R₅ is unsubstituted, mono- or disubstituted amino or a heterocyclic radical containing at least one nitrogen ring atom whereby the binding of the heterocyclic radical occurs via a nitrogen ring atom and Y is oxygen, a compound of formula I, in which R₂ is a radical R₅—(C═Y)—NH— wherein R₅ is imidazol-1-yl and Y is oxygen, is reacted with a compound of the formula R₅—H, in which R₅ is unsubstituted, mono- or disubstituted amino, or a heterocyclic radical which contains at least one nitrogen ring atom;
g) in order to prepare a compound of formula I, in which R₂ is a radical R₅—(C═Y)—NH— wherein R₅ is unsubstituted or monosubstituted amino and Y is oxygen or sulfur, a compound of formula I, in which R₂ is amino, is reacted with a compound of the formula R₁₂—N═C═Y wherein Y is oxygen or sulfur, the radical R₁₂—NH— corresponding to unsubstituted or monosubstituted amino R₅;
h) in order to prepare a compound of formula I, in which R₂ is a radical R₅—(C═Y)—NH— wherein R₅ is lower alkylamino wherein the lower alkyl moiety is substituted by unsubstituted, mono- or disubstituted amino or by a heterocyclic radical containing at least one nitrogen ring atom whereby the binding of the heterocyclic radical to the lower alkyl moiety occurs via a nitrogen ring atom and Y is oxygen or sulfur, a compound of formula VI 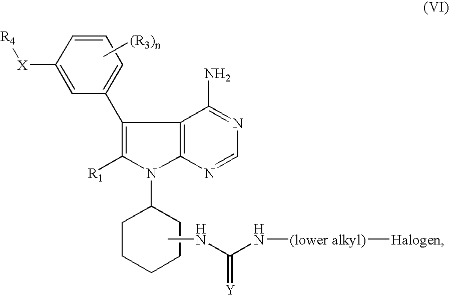
wherein Y is oxygen or sulfur and n, R₁, R₃, R₄ and X have the meanings as defined for a compound of formula I, is reacted with a compound of the formula R₁₃—H, in which R₁₃ is unsubstituted, mono- or disubstituted amino or a heterocyclic radical containing at least one nitrogen ring atom wherein the heterocyclic radical is attached to the hydrogen atom of R₁₃—H via a nitrogen ring atom;
i) in order to prepare a compound of formula I, in which R₂ is a radical R₅—(C═Y)—NH— wherein R₅ is etherified hydroxy and Y is oxygen, a compound of formula I, in which R₂ is amino, is reacted with a compound of the formula R₅—(C═O)-Halogen wherein R₅ is etherified hydroxy;
j) in order to prepare a compound of formula I, in which R₂ is a radical R₅—(C═Y)—NH— wherein R₅ is lower alkoxy substituted by unsubstituted, mono- or disubstituted amino or by a heterocyclic radical containing at least one nitrogen ring atom whereby the binding of the heterocyclic radical to the lower alkyl moiety of lower alkoxy occurs via a nitrogen ring atom and Y is oxygen, a compound of formula VII 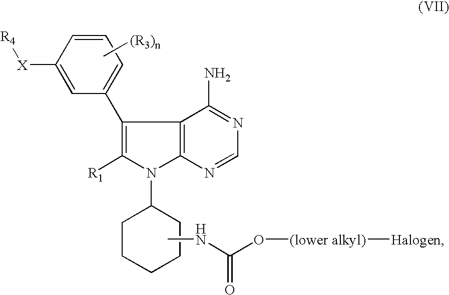
wherein n, R₁, R₃, R₄ and X have the meanings as defined for a compound of formula I, is reacted with a compound of the formula R₁₄—H, in which R₁₄ is unsubstituted, mono- or disubstituted amino or a heterocyclic radical containing at least one nitrogen ring atom wherein the heterocyclic radical is attached to the hydrogen atom of R₁₄—H via a nitrogen ring atom;
k) in order to prepare a compound of formula I, in which R₂ is a radical R₆-sulfonylamino wherein R₆ has the meanings as defined above under formula I, a compound of formula I, in which R₂ is amino, is reacted with R₆-sulfonyl halide;
l) in order to prepare a compound of formula I, in which R₁ is halogen, a compound of formula I, in which R₁ is hydrogen, is reacted with N-halosuccinimide;
m) in order to prepare a compound of formula I, in which R₁ is lower alkyl, a compound of formula I, in which R₁ is halogen, is reacted with tetra(lower alkyl) tin;
n) in order to prepare a compound of formula I, a compound of formula II, wherein n, R₁, R₃, R₄ and X have the meanings as defined for a compound of formula I, is reacted with a compound of formula VIII 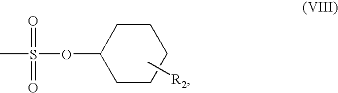
wherein R₂ has the meanings as defined for a compound of formula I;
wherein functional groups which are present in the starting compounds of processes a) to n) and are not intended to take part in the reaction, are present in protected form if necessary, and protecting groups that are present are cleaved, wherein said starting compounds may also exist in the form of salts provided that a salt-forming group is present and a reaction in salt form is possible,
and, if so desired, a compound of formula I thus obtained is converted into another compound of formula I, a free compound of formula I is converted into a salt, an obtained salt of a compound of formula I is converted into the free compound or another salt, and/or a mixture of isomeric compounds of formula I is separated into the individual isomers.
